(19) United States
(12) Patent Application Publication
(10) Pub. No.: US 2022/0135619 A1
(43) Pub. Date: May 5, 2022

(54) METHODS OF ISOLATING A PROTEIN

(71) Applicant: Bristol-Myers Squibb Company, Princeton, NJ (US)

(72) Inventors: Douglas D. FREY, Baltimore, MD (US); Yang LIU, Baltimore, MD (US); Sevda DELDARI, Baltimore, MD (US); Ryan Keith SWANSON, Carver, MN (US); Chittoor R. NARAHARI, Leominster, MA (US); Ronald Charles BATES, Irvine, CA (US); Sanchayita GHOSE, Acton, MA (US); Zhengjian LI, Sudbury, MA (US)

(73) Assignee: Bristol-Myers Squibb Company, Princeton, NJ (US)

(21) Appl. No.: 17/433,584

(22) PCT Filed: Feb. 24, 2020
(86) PCT No.: PCT/US2020/019472

§ 371 Date: Aug. 24, 2021

Related U.S. Application Data
(60) Provisional application No. 62/809,694, filed on Feb. 24, 2019.

(51) Int. Cl. C07K 1/36; C07K 1/28; C07K 1/18; C07K 1/20

(52) U.S. Cl. CPC C07K 1/36; C07K 1/20; C07K 1/18; C07K 1/28

(57) ABSTRACT

The disclosure provides a method of separating a protein product, e.g., from a contaminant in a mixture, comprising contacting the mixture with a polishing chromatography matrix. In some embodiments, the polishing chromatography matrix is used together with a retained gradient. In certain embodiments, the retained gradient comprises a pH gradient. In certain embodiments, the mixture comprises a monoclonal antibody product of a prior protein A chromatography.

### FIELD OF THE DISCLOSURE

The present disclosure provides a method for separating a protein product from one or more contaminants. In certain embodiments, the protein product is an antibody.

### BACKGROUND OF THE DISCLOSURE

As biologics move to the forefront of drug development, the need for improved protein purification and polishing has grown. The industrial purification of mAbs is most commonly performed using protein A affinity chromatography as the initial capture step followed by several polishing purification steps. Ion exchange chromatography has been widely used for the polishing step for mAbs purification, usually with either the flow-through mode or the bind and elute mode being performed. For the bind and elute chromatography mode, there is a capacity limit for the mAb product. The flow-through chromatography mode usually allows higher load challenges because the column packing capacity is only used to bind the impurities and not the product. But since conditions are set in a manner so that no substantial product is adsorbed, the impurities are typically adsorbed only weakly.

Weak partitioning chromatography as well as overload and elute chromatography have been introduced for the mAb purification of a protein A pool product (i.e., for “polishing”) (see, e.g., Kelley et al., Biotechnol. Bioeng., 101(3):553-566 (2008); Ishihara et al., MABS 10(2):325-334 (2018); Liu et al., J. Chromatogr. A 1218:6943-6952 (2011); Hill et al., J. Chromtogr. A523:11-21 (1990); Tsonev and Hirsh, J. Chromatogr. A 1200:166-182 (2008); US Publ. Nos. 2015/0133636 and 2007/0144973; WO 2013/067301 A1; and U.S. Pat. No. 8,067,182). (US). For both the traditional weak partitioning chromatography mode and the overload and elute chromatography mode, the mAb product partition coefficient (defined as the concentration of the mAb product in the stationary phase component of the column packing divided by the concentration of this product in the mobile phase at equilibrium and denoted as Kₚ) is higher than in the flow-through mode. This will generally mean that the impurities present will bind relatively strongly to the column packing and an enhanced purification will be obtained since these impurities typically have a partition coefficient that is larger than that of the mAb product. For weak partitioning chromatography, the mAb product Kₚ is in the range of 0.1-20 while for overload and elute chromatography the mAb product Kₚ is usually greater than 30 or even greater than 100 (which effectively ensures that nonlinear adsorption equilibrium applies). For the overload and elute chromatography mode, significantly more mAb product will remain on the column packing after the loading and washing step compared to the weak partitioning chromatography mode. Consequently, instead of washing out the bound product and impurities with a salt strip and discarding it as is the practice in weak partitioning chromatography, the bound mAb product for the overload and elute chromatography mode is recovered using an externally produced (and therefore unretained) pH or salt gradient with appropriately chosen conditions so that properly selected product fractions can be pooled.

The methods disclosed herein employ sequences of discrete self-sharpening pH fronts to accomplish the polishing purification of a protein product, e.g., a monoclonal antibody directly from the protein A pool, or from the protein A pool that has been subjected to low pH viral inactivation, pH neutralization and depth filtration.

### SUMMARY OF THE DISCLOSURE

Some aspects of the present disclosure are directed to a method of separating a protein product from a contaminant in a mixture, comprising contacting the mixture with a polishing chromatography matrix.

Some aspects of the present disclosure are directed to a method of polishing a protein product in a mixture comprising the protein product and a contaminant comprising contacting the mixture with a polishing chromatography matrix.

In some embodiments, the polishing chromatography matrix is used together with a retained gradient.

Some aspects of the present disclosure are directed to a method of separating a protein product from a contaminant in a mixture, comprising: (1) contacting the mixture with a polishing chromatography matrix, and (2) applying a retained gradient to the polishing chromatography matrix.

Some aspects of the present disclosure are directed to a method of polishing a protein product from a contaminant in a mixture, comprising (1) contacting the mixture with a polishing chromatography matrix, and (2) applying a retained gradient to the polishing chromatography matrix.

In some embodiments, the mixture is applied to the polishing chromatography matrix at a loading amount that is equal to the product dynamic binding capacity (DBC) of the polishing chromatography matrix. In some embodiments, the mixture is applied to the polishing chromatography matrix at a loading amount that is less than the product dynamic binding capacity (DBC) of the polishing chromatography matrix. In some embodiments, the mixture is applied to the polishing chromatography matrix at a loading amount that exceeds the dynamic binding capacity (DBC) of the polishing chromatography matrix.

In some embodiments, the protein product comprises a monoclonal antibody or an antigen binding fragment thereof (“mAb”) or an Fc fusion protein. In some embodiments, the contaminant associates with the polishing chromatography matrix.

In some embodiments, the retained gradient comprises a pH gradient. In some embodiments, the retained gradient comprises a retained ionic strength gradient.

In some embodiments, the loading amount of the mixture that is applied to the polishing chromatography matrix is at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold higher than the DBC of the polishing chromatography matrix.

In some embodiments, the mixture comprises a product of a prior purification step. In some embodiments, the prior purification step comprises applying a feed material to the prior purification step, wherein the feed material comprises a cell culture harvest, wherein the cell culture harvest comprises the protein product. In some embodiments, the prior purification step comprises a protein bind and elute chromatography. In some embodiments, the prior purification step comprises an anion-exchange chromatography, a cation-exchange chromatography, an affinity chromatography, a mixed mode chromatography, a hydrophobic-interaction chromatography or any combination thereof. In some embodiments, the prior purification step comprises a protein A chromatography.

In some embodiments, the polishing chromatography matrix comprises an anion-exchange matrix, a cation-exchange matrix, a mixed-mode matrix, a hydrophobic-interaction chromatography matrix or any combination thereof. In some embodiments, the polishing chromatography matrix comprises an anion-exchange matrix.

In some embodiments, a starting buffer is applied to the polishing chromatography matrix. In some embodiments, the starting buffer is applied to the polishing chromatography matrix prior to the contacting of the mixture comprising the protein product and the contaminant with the polishing chromatography matrix. In some embodiments, the starting buffer comprises N-(1,1-Dimethyl-2-hydroxyethyl)-3-amino-2-hydroxypropanesulfonic acid (AMPSO), 3-morpholin-4-ylpropane-1-sulfonic acid (MOPS), or both.

In some embodiments, the starting buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM AMPSO. In some embodiments, the starting buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, or about 10 mM to about 15 mM AMPSO. In some embodiments, the starting buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO. In some embodiments, the starting buffer comprises about 9.4 mM AMPSO.

In some embodiments, the starting buffer comprises at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, or at least about 30 mM MOPS. In some embodiments, the starting buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS. In some embodiments, the starting buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS. In some embodiments, the starting buffer comprises about 20.5 mM MOPS.

In some embodiments, the starting buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10. In some embodiments, the starting buffer has a pH of about 9.2.

In some embodiments, the starting buffer comprises more than one buffering species, wherein each of the more than one buffering species travels through the matrix at a different velocity, thereby establishing a retained gradient. In some embodiments, the pH of the retained gradient is determined based on the apparent isoelectric points of the protein product and the contaminant. In some embodiments, the apparent isoelectric points of the protein product and the contaminant are determined using a 2D analytical fractionation process. In some embodiments, the 2D analytical fractionation process comprises subjecting the mixture comprising the protein product and the contaminant to an analytical chromatography using a retained gradient with multiple pH fronts followed by high-sensitivity silver stained SDS-PAGE.

In some embodiments, the method further comprising washing the polishing chromatography matrix with a wash buffer after the mixture comprising the protein product and the contaminant is contacted with the polishing chromatography matrix. In some embodiments, the wash buffer comprises AMPSO, MOPS, or both.

In some embodiments, the wash buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM AMPSO. In some embodiments, the wash buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, or about 10 mM to about 15 mM AMPSO. In some embodiments, the wash buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO. In some embodiments, the wash buffer comprises about 9.4 mM AMPSO.

In some embodiments, the wash buffer comprises at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, or at least about 30 mM MOPS. In some embodiments, the wash buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS. In some embodiments, the wash buffer comprises about 19.0 mM, about 19.1 mM, about 19.2 mM, about 19.3 mM, about 19.4 mM, about 19.5 mM, about 19.6 mM, about 19.7 mM about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS. In some embodiments, the wash buffer comprises about 20.5 mM MOPS.

In some embodiments, the wash buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10. In some embodiments, the wash buffer has a pH of about 9.2.

In some embodiments, the wash buffer comprises more than one buffering species, wherein each of the more than one buffering species travels through the matrix at a different velocity, thereby establishing a retained gradient.

In some embodiments, the wash buffer is the same as the starting buffer.

In some embodiments, the retained gradient is created by performing a stepwise change between the starting buffer and an elution buffer. In some embodiments, the elution buffer comprises MOPS, 4-Morpholineethanesulfonic acid monohydrate (MES), or both.

In some embodiments, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM MOPS. In some embodiments, the elution buffer comprises about 0.1 mM to about 20 mM, about 0.5 mM to about 15 mM, about 5 mM to about 15 mM, about 6 mM to about 15 mM, about 7 mM to about 15 mM, about 8 mM to about 15 mM, about 9 mM to about 15 mM, about 10 mM to about 15 mM, about 10 mM to about 14 mM, or about 11 mM to about 13 mM MOPS. In some embodiments, the elution buffer comprises about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, or about 13.0 mM MOPS. In some embodiments, the elution buffer comprises about 12 mM MOPS.

In some embodiments, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM MES. In some embodiments, the elution buffer comprises about 0.1 mM to about 20 mM, about 0.5 mM to about 15 mM, about 5 mM to about 15 mM, about 6 mM to about 14 mM, about 7 mM to about 13 mM, about 8 mM to about 12 mM, about 9 mM to about 11 mM, about 5 mM to about 10 mM, or about 10 mM to about 15 mM MES. In some embodiments, the elution buffer comprises about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10.0 mM, about 10.1 mM, about 10.2 mM, about 10.3 mM, about 10.4 mM, about 10.5 mM, about 10.6 mM, about 10.7 mM, about 10.8 mM, about 10.9 mM, or about 11.0 mM MES. In some embodiments, the elution buffer comprises about 10 mM MES.

In some embodiments, the elution buffer has a pH of less than about 8.0, less than about 7.9, less than about 7.8, less than about 7.7, less than about 7.6, less than about 7.5, less than about 7.4, less than about 7.3, less than about 7.2, less than about 7.1, less than about 7.0, less than about 6.9, less than about 6.8, less than about 6.7, less than about 6.6, less than about 6.5, less than about 6.4, less than about 6.3, less than about 6.2, less than about 6.1, less than about 6.0, less than about 5.9, less than about 5.8, less than about 5.7, less than about 5.6, less than about 5.5, less than about 5.4, less than about 5.3, less than about 5.2, or less than about 5.1.

In some embodiments, the elution buffer has a pH of about 6.2. In some embodiments, the elution buffer comprises more than one buffering species, wherein the more than one buffering species flow through the matrix at different velocities, thereby establishing a retained gradient. In some embodiments, at least one of the more than one buffering species of the elution buffer is different from the more than one buffering species of the starting buffer.

In some embodiments, the starting buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min. In some embodiments, the starting buffer is applied to the polishing chromatography matrix at a volumetric flow rate corresponding to a superficial velocity of about 5.0 cm/min.

In some embodiments, the starting buffer is applied to the polishing chromatography matrix for about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, or about 50 minutes. In some embodiments, the starting buffer is applied to the polishing chromatography matrix for about 40 minutes.

In some embodiments, the wash buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min. In some embodiments, the wash buffer is applied to the polishing chromatography matrix at a volume flow rate corresponding to a superficial velocity of about 5.0 cm/min.

In some embodiments, the wash buffer is applied to the polishing chromatography matrix for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, 21 minute, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes. In some embodiments, the wash buffer is applied to the polishing chromatography matrix for about 10 minutes. In some embodiments, the wash buffer is applied to the polishing chromatography matrix for about 30 minutes.

In some embodiments, the stepwise change between the starting buffer and the elution buffer is applied to the polishing chromatography matrix at a volumetric flow rate corresponding to a superficial liquid velocity of at least about 1.4 cm/min, at least about 1.5 cm/min, at least about 1.6 cm/min, at least about 1.7 cm/min, at least about 1.8 cm/min, at least about 1.9 cm/min, at least about 2.0 cm/min, at least about 2.1 cm/min, at least about 2.2 cm/min, at least about 2.3 cm/min, at least about 2.4 cm/min, at least about 2.5 cm/min, at least about 2.6 cm/min, at least about 2.7 cm/min, at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min or at least about 4.0 cm/min. In some embodiments, the stepwise change between the starting buffer and the elution buffer is applied to the polishing chromatography matrix at a volume flow rate corresponding to a superficial velocity of about 2.8 cm/min.

In some embodiments, the method further comprises a second polishing chromatography matrix, wherein material eluted from the polishing chromatography matrix following the contacting of the mixture with the polishing chromatography column matrix is applied to a second polishing chromatography matrix.

In some embodiments, a column switching method is employed for overload and elution after the mixture is contacted with the polishing chromatography matrix, wherein an auxiliary column containing a second polishing chromatography matrix is inserted into the mobile phase flow path downstream from the polishing chromatography matrix during elution.

### BRIEF DESCRIPTION OF THE DRAWINGS

FIGS. 1A, 1C, and 1D are graphical representations of proteins in a retained pH gradient when the band velocity curves of three proteins intersect different pH fronts (FIG. 1A), when the band velocity curves of two proteins intersect the same pH front under dilute conditions (FIG. 1C) and under mass overloaded conditions (FIG. 1D). FIG. 1B is a drawing illustrating a descending pH gradient.

FIG. 2A is a schematic of a 2D analytical tool for polishing process development in anion-exchange chromatography methods. FIGS. 2B-2D are graphical representations of computer aided parameter fitting (FIGS. 2B-2C) and buffer design (FIG. 2D) for chromatofocusing based on local equilibrium theory as a tool for rational process development.

FIG. 3 is a schematic drawing of the protein concentration profiles for weak partitioning chromatography. mAb=monoclonal antibody; HCP=host cell proteins.

FIG. 4 is a schematic drawing of the protein concentration profiles for an anion-exchange chromatography for the polishing of a monoclonal antibody in the overload and elute chromatography mode, wherein a retained pH gradient is used.

FIGS. 5A-5H are graphical representations showing UV absorbance (FIGS. 5A, 5C, 5E, 5G and 5H) and SDS-PAGE (FIGS. 5B, 5D, and 5F) results from a 2D analytical fractionation process development tool applied to monoclonal antibody protein A pools. Two antibodies were used, antibody B (FIGS. 5A-5F) and antibody D (FIGS. 5G-5H). A Super Q column packing (FIGS. 5A, 5B, and 5E-5G) or GigaCap column packing (FIGS. 5C, 5D, and 5H) was used. The samples used were either a protein A pool after low pH virus inactivation, pH neutralization, and depth filtration, defined as sample type #1 (FIGS. 5A, 5B, 5C, 5D, 5G, and 5H), or a protein A pool, defined as sample type #2 (FIGS. 5E and 5F).

FIG. 6A is a schematic drawing of an anion-exchange chromatography method for the polishing of a monoclonal antibody in the bind-and-elute mode. FIG. 6B is a graphical representation showing the UV absorbance at 280 nm of the flow through and effluent following anion-exchange chromatography for the polishing of a monoclonal antibody in the bind and elute mode using a Super Q anion-exchanger column. The monoclonal antibodies were obtained from sample type #1, antibody B protein A pools. FIG. 6C is a graphical representation showing the UV absorbance at 280 nm of the flow through and effluent following anion-exchange chromatography for the polishing of a monoclonal antibody in the bind and elute mode using a Super Q anion-exchanger column. The monoclonal antibodies were obtained from sample type #2, antibody B protein A pools. FIG. 6D is a graphical representation showing the UV absorbance at 280 nm of the flow through and effluent following anion-exchange chromatography for the polishing of a monoclonal antibody in the bind and elute mode using a GigaCap anion-exchanger column. The monoclonal antibodies were obtained from sample type #1, antibody B protein A pools. FIG. 6E is a graphical representation showing the UV absorbance at 280 nm of the flow through and effluent following anion-exchange chromatography for the polishing of a monoclonal antibody in the bind and elute mode using a GigaCap anion-exchanger column. The monoclonal antibodies were obtained from sample type 2, antibody B protein A pools. FIG. 6F is a graphical representation showing the UV absorbance at 280 nm of the flow through and effluent following anion-exchange chromatography for the polishing of a monoclonal antibody in the bind and elute mode using a GigaCap anion-exchanger column. The monoclonal antibodies were obtained from sample type #1, antibody D protein A pools. FIG. 6G is a graphical representation showing the UV absorbance at 280 nm of the flow through and effluent following cation-exchange chromatography for the polishing of a monoclonal antibody in the bind and elute mode using a Fractogel SO₃⁻ cation-exchanger column. The monoclonal antibodies were obtained from sample type #1, antibody B protein A pools. The green box indicates the main collection (FIGS. 6B-6G).

FIG. 7A is a schematic drawing of the overload-and-elute mode of anion-exchange chromatography for the polishing of monoclonal antibodies from protein A pools. FIG. 7B is a graphical representation showing the UV absorbance at 280 nm of the flow through and effluent following anion-exchange chromatography for the polishing of a monoclonal antibody in the overload and elute mode using a Super Q anion-exchanger column. The monoclonal antibodies were obtained from sample type #1, antibody B protein A pools. FIG. 7C is a graphical representation showing the UV absorbance at 280 nm of the flow through and effluent following anion-exchange chromatography for the polishing of a monoclonal antibody in the overload and elute mode using a Super Q anion-exchanger column. The monoclonal antibodies were obtained from sample type #2, antibody B protein A pools. FIG. 7D is a graphical representation showing the high molecular weight proteins (HMWs) resulting from the anion-exchange chromatography method of FIG. 7B. FIG. 7E is a graphical representation showing the relative HCPs (host cell proteins) resulting from the anion-exchange chromatography method of FIG. 7B. FIG. 7F is a graphical representation showing the HMWs resulting from the anion-exchange chromatography method of FIG. 7C. FIG. 7G is a graphical representation showing relative HCPs (host cell proteins) resulting from the anion-exchange chromatography method of FIG. 7C. FIG. 7H is a graphical representation showing the UV absorbance at 280 nm of the flow through and effluent following anion-exchange chromatography for the polishing of a monoclonal antibody in the overload and elute mode using a GigaCap anion-exchanger column. The monoclonal antibodies were obtained from sample type #1 antibody D protein A pools. FIG. 7I is a graphical representation showing the HMWs resulting from the anion-exchange chromatography method of FIG. 7H.

FIG. 8A is a schematic drawing of the overload-and-elute mode of anion-exchange chromatography using two anion-exchanger columns with column switching for the polishing of monoclonal antibodies from protein A pools. FIG. 8B is a graphical representation showing the UV absorbance at 280 nm of the flow through and effluent following anion-exchange chromatography for the polishing of a monoclonal antibody in the overload and elute mode using two equal size Super Q anion-exchanger columns with column switching. The monoclonal antibodies were obtained from sample type #1, antibody B, protein A pools. FIG. 8C is a graphical representation showing the UV absorbance at 280 nm of the flow through and effluent following anion-exchange chromatography for the polishing of a monoclonal antibody in the overload and elute mode using two equal size Super Q anion-exchanger columns with column switching. The monoclonal antibodies were obtained from sample type #2, antibody B, protein A pools. FIG. 8D is a graphical representation showing the HMWs resulting from the anion-exchange chromatography method of FIG. 8B. FIG. 8E is a graphical representation showing the relative HCPs resulting from the anion-exchange chromatography method of FIG. 8B. FIG. 8F is a graphical representation showing the HMWs resulting from the anion-exchange chromatography method of FIG. 8C. FIG. 8G is a graphical representation showing the relative HCPs resulting from the anion-exchange chromatography method of FIG. 8C. FIG. 8H is a graphical representation showing the UV absorbance at 280 nm of the flow through and effluent following anion-exchange chromatography for the polishing of a monoclonal antibody in the overload and elute mode using two equal size GigaCap anion-exchanger columns with column switching. The monoclonal antibodies were obtained from sample type #1 antibody D protein A pools. FIG. 8I is a graphical representation showing the HMWs resulting from the anion-exchange chromatography method of FIG. 8H.

FIG. 9A is a schematic drawing of a comparative method of overload-and-elute mode of anion-exchange chromatography using two anion-exchanger columns without column switching for the polishing of monoclonal antibodies from protein A pools. FIG. 9B is a graphical representation showing the UV absorbance at 280 nm of the flow through and effluent following anion-exchange chromatography for the polishing of a monoclonal antibody in the overload and elute mode using two equal size Super Q anion-exchanger columns without column switching. The monoclonal antibodies were obtained from sample type #1, antibody B, protein A pools. FIG. 9C is a graphical representation showing the UV absorbance at 280 nm of the flow through and effluent following anion-exchange chromatography for the polishing of a monoclonal antibody in the overload and elute mode using two equal size Super Q anion-exchanger columns without column switching. The monoclonal antibodies were obtained from sample type #2, antibody B, protein A pools. FIG. 9D is a graphical representation showing the HMWs resulting from the anion-exchange chromatography method of FIG. 9B. FIG. 9E is a graphical representation showing the relative HCPs resulting from the anion-exchange chromatography method of FIG. 9B. FIG. 9F is a graphical representation showing the HMWs resulting from the anion-exchange chromatography method of FIG. 9C. FIG. 9G is a graphical representation showing the relative HCPs resulting from the anion-exchange chromatography method of FIG. 9C. FIG. 9H is a graphical representation showing the UV absorbance at 280 nm of the flow through and effluent following anion-exchange chromatography for the polishing of a monoclonal antibody in the overload and elute mode using two equal size GigaCap anion-exchanger columns without column switching. The monoclonal antibodies were obtained from sample type #1 antibody D protein A pools. FIG. 9I is a graphical representation showing the HMWs resulting from the anion-exchange chromatography method of FIG. 9H.

### DETAILED DESCRIPTION OF THE DISCLOSURE

The present disclosure is directed to methods of separating a protein product from a contaminant in a mixture, comprising contacting the mixture with a polishing chromatography matrix. In some embodiments, the polishing chromatography matrix is used together with a retained gradient, e.g., a retained pH gradient.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “chromatography,” as used herein, refers to a dynamic separation technique which separates a target molecule such as a target protein (e.g., an immunoglobulin or another Fc-containing protein) from other molecules in the mixture and allows it to be isolated. Typically, in a chromatography method, a liquid mobile phase transports a sample containing the target molecule of interest across or through a stationary phase (normally solid) medium. Differences in partition or affinity to the stationary phase causes the temporary binding of selected molecules to the stationary phase while the mobile phase carries different molecules out at different times.

The term “contacting” as used herein, refers to applying a solution, e.g., a mixture comprising a protein product and a contaminant, as described herein, to a chromatography matrix. In some embodiments, the term “contacting” is synonymous with “loading” a solution onto a chromatography column. A “column packing” or a “chromatography matrix” as used herein refers to the adsorbent solid material contained within a chromatography column. In some aspects, the column packing comprises Super Q. In some aspects, the column packing comprises GigaCap. In some aspects, the column packing comprises FRACTOGEL® SO₃⁻.

The term “is applied to,” when used in the context of a gradient being applied to a chromatography matrix, broadly means that a gradient is formed, directly or indirectly, within and/or around a chromatography matrix. In some embodiments, the chromatography matrix is present in a column, and the gradient is formed within the column. In some embodiments, a gradient that is applied to a chromatography matrix is formed internally within a column, as opposed to a gradient which is formed externally and then added to a column. In certain embodiments, a gradient that is applied to the chromatography matrix forms within a column as a result of more than one buffer being added to the chromatography matrix. In other embodiments, a gradient that is applied to the chromatography matrix is formed externally and then added to the column.

“Superficial velocity of liquid,” or “superficial velocity of fluid,” or “superficial velocity of buffer,” as used herein, refers to the volumetric flow rate (in units of volume per time) divided by the column cross-sectional area (in units of area). The units of superficial velocity are length per time. The superficial velocity of the liquid is the hypothetical steady-state velocity of the liquid for a given steady-state volumetric flow rate if the column was empty and contained no column packing and if there was no spatial variation in fluid velocity in the column.

The term “affinity chromatography,” as used herein, refers to a mode of chromatography where a target molecule, such as a protein molecule (e.g., an Fc-containing protein) to be separated is isolated by its “lock-and-key” interaction with a molecule (e.g., a Protein A based ligand) immobilized on the chromatography resin. This specific interaction allows the target molecule to bind the molecule immobilized on the resin while the undesirable molecules flow through. Changing the temperature, pH, or ionic strength of the mobile phase then releases the target molecule in high purity. In various embodiments described herein, affinity chromatography involves the addition of a sample containing a target molecule (e.g., an immunoglobulin or another Fc-containing protein) to a solid support which carries on it a ligand based on the C domain of Protein A (referred to as Protein A affinity chromatography media or resin). Other ligands used for affinity chromatography can include, e.g., Protein G from Steptococci which binds to the Fc region of a immunoglobulin.

The term “Protein A affinity chromatography” or “Protein A chromatography,” as used herein, refers to the separation or isolation of substances using Protein A, a protein found in the cell wall of the bacterium Staphylococcous aureus, that is capable of binding to immunoglobulins at the Fc region. Protein A is immobilized on a solid support and is contacted with the protein of interest.

The term “solid support” refers in general to any resin (porous or non-porous) to which a ligand is attached. The attachment of ligands to the solid support can either be through a covalent bond, such as in the case of grafting (via ether, thioether, carbon-carbon bond, or other linkages), or through coating, adhesion, adsorption, and similar mechanisms. Exemplary solid supports used in the methods described herein include polyvinylether, polyvinylalcohol, polymethacrylate, polyacrylate, polystyrene, polyacrylamide, polymethacrylamide and polycarbonate.

Examples of Protein A affinity chromatography resins known in the art include those having the Protein A immobilized onto a controlled pore glass backbone, e.g., PROSEP™ media/resin (EMD MILLIPORE); those having Protein A immobilized onto a polystyrene solid phase, e.g., the POROS™ MabCaptureTMA media/resin (APPLIED BIOSYSTEMS INC.); and those having Protein A immobilized on an agarose solid support, e.g., rPROTEIN A SEPHAROSE FAST FLOW™ or MABSELEC™ media or resins (GE HEALTHCARE).

The term “affinity resin” or “affinity chromatography resin” or “affinity media” or “affinity chromatography media,” as used interchangeably herein, refers to an affinity chromatography ligand (e.g., based on Protein A) attached to a solid support such as, e.g., those described herein. In general, the terms “resin” and “media” are used interchangeably herein. Other examples of affinity chromatography resin include resin with Protein G from Streptococci capable of binding to the Fc region of immunoglobulins. Also included are affinity resins with Protein L which binds immunoglobulins through the kappa light chain. These proteins can be used in an affinity chromatography to purify immunoglobulins or other proteins which bind specifically to these affinity resins.

The term “bind and elute” chromatography, as used herein, refers to a form or mode of chromatography wherein a feed sample is loaded close to the product dynamic binding capacity (DBC) of a chromatography matrix, e.g., the column packing, without exceeding the DBC of the chromatography matrix. In doing so, the entirety of the feed sample is retained in the chromatography matrix, e.g., no portion of the feed sample is eluted during the loading phased. In some embodiments, a wash step is performed after loading. In some embodiments, a target protein, e.g., a protein product described herein, is eluted from the bind and elute chromatography matrix using a single stepwise change from the starting buffer to the elution buffer. In certain embodiments, the elution comprises formation of a retained pH gradient, wherein most or all of the antibody product is eluted in a separate pH front, e.g., within one or two pH fronts, than the contaminants, which are eluted in subsequent pH fronts or which remain adsorbed on the column.

The term “overload and elute” chromatography, as used herein, refers to a form or mode of chromatography wherein a feed sample is loaded in excess of the DBC of a chromatography matrix, e.g., the column packing. In some embodiments, the feed material is loaded until impurities start to break through in the column effluent. In some embodiments, a wash step is performed, e.g., with a loading buffer or a wash buffer described herein. In certain embodiments, the wash step is performed until the UV absorbance returns to near baseline level. Although the product can start to break through in the column effluent at some point after continually loading the feed material, most impurities continue to bind during the breakthrough phase. Consequently, the product pool collected during the load phase is highly pure when using an overload and elute mode described herein. The remaining product bound onto the column can be recovered in selected effluent fractions by eluting with a chromatofocusing pH gradient described herein, while at the same time most of the impurities will either elute in different effluent fractions or will remain bound to the column packing so that they can be washed out of the column with a high salt buffer after the chromatofocusing step.

The term “flow-through” chromatography, as used herein, refers to a form of chromatography, wherein a chromatography matrix is used that is not intended to retain or specifically bind with the target protein, e.g., the protein product (e.g., the monoclonal antibody). Under “flow-through” chromatography, a mixture comprising the target protein is applied to the chromatography matrix, and the effluent comprises the target protein.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies and Fc domain-containing polypeptides (e.g., immunoadhesins).

The term “target protein” or “protein product” as used interchangeably herein, refers to any protein that can be purified using the polishing chromatography matrix. In various embodiments, the protein product is an antibody product, e.g., an antibody isolated through Protein A chromatography. In other embodiments, the protein product is an Fc fusion protein. In some embodiments, the protein product is a naturally-occurring protein. In other embodiments, the protein product is a recombinant protein.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The term “antigen-binding portion” or “antigen-binding fragment” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “focusing,” as used herein, refers to the effect where a species prefers to locate itself at a specific position on a mobile phase gradient.

The term “isocratic,” as used herein, refers to a chromatographic method where the mobile phase composition (and therefore the “eluting strength”) inside the column does not change with respect to time or position.

The term “mass overload” or “mass overloaded,” as used herein, refers to the condition where sufficient adsorbing species are present so that a significant fraction of the column packing adsorption capacity is utilized and the corresponding adsorption isotherms are therefore nonlinear.

The term “mobile phase,” as used herein refers to the phase that moves through the chromatography matrix. In the current context the mobile phase is the liquid phase.

The term “protein band velocity curve,” as used herein, refers to the functional relation at a fixed ionic strength between the velocity of a trace protein band and the mobile phase pH. The term “trace,” as used herein, denotes that the protein is present at low concentrations.

The term “polishing,” as used herein, refers to the subsequent (or at least a second stage) purification of a material recovered from a bioreactor cell culture harvest using a first-stage (or “capture”) purification method.

The term “retained gradient” or “composition front,” as used herein, refers to a spatial variation in elution buffer composition (and therefore of pH and/or ionic strength) that travels through the column more slowly than the velocity of an inert component that does not interact with the column packing material. This spatial variation is termed a “gradient.” “front,” or “wave.”

The term “self-generated gradient” or “self-generated composition front,” as used herein, refers to a spatial variation in mobile phase composition (and therefore of pH) that is formed inside the column as a result of the adsorption equilibrium processes that apply, as opposed to a gradient (commonly termed an external gradient) that is first formed outside the column by a gradient mixing device and then subsequently introduced into the column.

A “self-sharpening front,” as used herein, refers to a spatial variation in an elution buffer composition (and therefore in pH) or in a protein composition that does not broaden in its spatial distribution as time increases. Such fronts are typically steep enough in their spatial distribution at a given time to be considered stepwise in nature.

An “unretained gradient or composition front,” as used herein, is a spatial variation in elution buffer composition (and therefore of pH) that travels through the column at the same velocity as an inert component that does not interact with the column packing material. In certain embodiments, unretained gradients are generated by external means and are not internally generated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range, or integer range is to be understood to include the value of any integer within the recited range and, when appropriate, fractions thereof (such as one tenth and one hundredth of an integer), unless otherwise indicated.

Technical terms in addition to those given above that are used herein are defined in the article “Glossary of HPLC/LC separation terms,” by P. W. Carr and R. E. Majors (LCGC NorthAmerica 26(2):118-168 (2008)). This article is available online at www.chromatographyonline.com/glossary-hplclc-separation-terms.

### II. Methods of the Disclosure

The present disclosure is directed to methods of separating a protein product from a contaminant in a mixture, comprising contacting the mixture with a polishing chromatography matrix. Certain aspects of the present disclosure are directed to a method of polishing a protein product in a mixture comprising the protein product and a contaminant, comprising contacting the mixture with a polishing chromatography matrix. In certain embodiments, the polishing chromatography matrix is used together with a retained gradient. In some embodiments, a retained gradient is applied to the polishing chromatography matrix. In certain embodiments, the mixture is applied to the polishing chromatography matrix at a loading amount that is less than or equal to the product dynamic binding capacity (DBC) of the polishing chromatography matrix, e.g., in bind and elute mode. In some embodiments, the mixture is applied to the polishing chromatography matrix at a loading amount that exceeds the dynamic binding capacity (DBC) of the polishing chromatography matrix, e.g., in overload and elute mode.

Some aspects of the present disclosure are directed to a method of separating a protein product from a contaminant in a mixture, comprising: (1) contacting the mixture with a polishing chromatography matrix, and (2) applying a retained gradient to the polishing chromatography matrix. Some aspects of the present disclosure are directed to a method of polishing a protein product from a contaminant in a mixture, comprising: (1) contacting the mixture with a polishing chromatography matrix, and (2) applying a retained gradient to the polishing chromatography matrix.

I.A. Polishing Chromatography Matrix

Certain aspects of the present disclosure are directed to methods of separating a protein product from a contaminant in a mixture comprising contacting the protein product with a polishing chromatography matrix.

In some embodiments, the polishing chromatography matrix comprises an ion-exchange chromatography matrix. In some embodiments, the ion-exchange chromatography matrix is an anion-exchange chromatography matrix. In some embodiments, the ion-exchange chromatography matrix is a cation-exchange chromatography matrix. In certain embodiments, the polishing chromatography matrix comprises a mixed-mode chromatography (MMC) matrix. In certain embodiments, the polishing chromatography matrix comprises a hydrophobic-interaction chromatography matrix. In certain embodiments, the polishing chromatography matrix comprises TOYOPEARL SuperQ-650S. In some aspects, the polishing chromatography matrix comprises GigaCap. In some aspects, the polishing chromatography matrix comprises FRACTOGEL® SO₃⁻.

In some embodiments, the polishing chromatography matrix does not interact directly with the protein product. In some embodiments, the polishing chromatography matrix interacts with the contaminant.

In some embodiments, the methods disclosed herein comprise applying a starting buffer to the polishing chromatography matrix. In certain embodiments, the starting buffer is applied to the polishing chromatography matrix prior to the contacting of the mixture comprising the protein product and the contaminant with the polishing chromatography matrix. In some embodiments, the starting buffer is applied to the polishing chromatography matrix at the same time as the contacting of the mixture comprising the protein product and the contaminant with the polishing chromatography matrix. In certain embodiments, the mixture comprising the protein product and the contaminant is reconstituted in the starting buffer. In certain embodiments, the mixture comprising the protein product and the contaminant is diluted in the starting buffer. In other embodiments, the starting buffer is added after the contacting of the mixture comprising the protein product and the contaminant with the polishing chromatography matrix.

In some embodiments, the starting buffer comprises N-(1,1-Dimethyl-2-hydroxyethyl)-3-amino-2-hydroxypropanesulfonic acid (AMPSO). In some embodiments, the starting buffer comprises 3-morpholin-4-ylpropane-1-sulfonic acid (MOPS), or both. In some embodiments, the starting buffer comprises AMPSO and MOPS.

In certain embodiments, the starting buffer comprises at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM AMPSO. In some embodiments, the starting buffer comprises at least about 5 mM AMPSO. In some embodiments, the starting buffer comprises at least about 6 mM AMPSO. In some embodiments, the starting buffer comprises at least about 7 mM AMPSO. In some embodiments, the starting buffer comprises at least about 8 mM AMPSO. In some embodiments, the starting buffer comprises at least about 9 mM AMPSO. In some embodiments, the starting buffer comprises at least about 10 mM AMPSO. In some aspects, the starting buffer comprises at least about 11 mM AMPSO. In some aspects, the starting buffer comprises at least about 12 mM AMPSO. In some aspects, the starting buffer comprises at least about 13 mM AMPSO. In some aspects, the starting buffer comprises at least about 14 mM AMPSO. In some aspects, the starting buffer comprises at least about 15 mM AMPSO. In some aspects, the starting buffer comprises at least about 16 mM AMPSO. In some aspects, the starting buffer comprises at least about 17 mM AMPSO. In some aspects, the starting buffer comprises at least about 18 mM AMPSO. In some aspects, the starting buffer comprises at least about 19 mM AMPSO. In some aspects, the starting buffer comprises at least about 20 mM AMPSO. In some aspects, the starting buffer comprises at least about 20.5 mM AMPSO. In some aspects, the starting buffer comprises at least about 21 mM AMPSO. In some aspects, the starting buffer comprises at least about 22 mM AMPSO. In some aspects, the starting buffer comprises at least about 23 mM AMPSO. In some aspects, the starting buffer comprises at least about 24 mM AMPSO. In some aspects, the starting buffer comprises at least about 25.

In certain embodiments, the starting buffer comprises about 0.1 mM to about 25 mM, about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, about 10 mM to about 15 mM AMPSO, about 10 mM to about 20 mM AMPSO, about 10 mM to about 25 mM AMPSO, about 15 mM to about 20 mM AMPSO, about 15 mM to about 25 mM AMPSO, or about 20 mM to about 25 mM AMPSO. In some embodiments, the starting buffer comprises about 5 mM to about 15 mM AMPSO. In some embodiments, the starting buffer comprises about 5 mM to about 10 mM AMPSO. In some embodiments, the starting buffer comprises about 6 mM to about 10 mM AMPSO. In some embodiments, the starting buffer comprises about 7 mM to about 10 mM AMPSO. In some embodiments, the starting buffer comprises about 8 mM to about 10 mM AMPSO. In some embodiments, the starting buffer comprises about 9 mM to about 10 mM AMPSO. In some aspects, the starting buffer comprises about 10 mM to about 15 mM AMPSO. In some aspects, the starting buffer comprises about 10 mM to about 20 mM AMPSO. In some aspects, the starting buffer comprises about 10 mM to about 25 mM AMPSO. In some aspects, the starting buffer comprises about 15 mM to about 20 mM AMPSO. In some aspects, the starting buffer comprises about 15 mM to about 25 mM AMPSO. In some aspects, the starting buffer comprises about 20 mM to about 25 mM AMPSO.

In certain embodiments, the starting buffer comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 20.5 mM, about 21 mM, about 22 mM, about 23 mM, about 24 mM, or about 25 AMPSO. In some embodiments, the starting buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO. In some aspects, the starting buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 AMPSO. In certain embodiments, the starting buffer comprises about 8 mM AMPSO. In certain embodiments, the starting buffer comprises about 8.5 mM AMPSO. In certain embodiments, the starting buffer comprises about 8.6 mM AMPSO. In certain embodiments, the starting buffer comprises about 8.7 mM AMPSO. In certain embodiments, the starting buffer comprises about 8.8 mM AMPSO. In certain embodiments, the starting buffer comprises about 8.9 mM AMPSO. In certain embodiments, the starting buffer comprises about 9 mM AMPSO. In certain embodiments, the starting buffer comprises about 9.1 mM AMPSO. In certain embodiments, the starting buffer comprises about 9.2 mM AMPSO. In certain embodiments, the starting buffer comprises about 9.3 mM AMPSO. In certain embodiments, the starting buffer comprises about 9.4 mM AMPSO. In certain embodiments, the starting buffer comprises about 9.5 mM AMPSO. In certain embodiments, the starting buffer comprises about 9.6 mM AMPSO. In certain embodiments, the starting buffer comprises about 9.7 mM AMPSO. In certain embodiments, the starting buffer comprises about 9.8 mM AMPSO. In certain embodiments, the starting buffer comprises about 9.9 mM AMPSO. In certain embodiments, the starting buffer comprises about 10 mM AMPSO. In certain embodiments, the starting buffer comprises about 10.1 mM AMPSO. In certain embodiments, the starting buffer comprises about 10.2 mM AMPSO. In certain embodiments, the starting buffer comprises about 10.3 mM AMPSO. In certain embodiments, the starting buffer comprises about 10.4 mM AMPSO. In certain embodiments, the starting buffer comprises about 10.5 mM AMPSO. In certain aspects, the starting buffer comprises about 19.5 mM AMPSO. In certain aspects, the starting buffer comprises about 19.6 mM AMPSO. In certain aspects, the starting buffer comprises about 19.7 mM AMPSO. In certain aspects, the starting buffer comprises about 19.8 mM AMPSO. In certain aspects, the starting buffer comprises about 19.9 mM AMPSO. In certain aspects, the starting buffer comprises about 20 mM AMPSO. In certain aspects, the starting buffer comprises about 20.1 mM AMPSO. In certain aspects, the starting buffer comprises about 20.2 mM AMPSO. In certain aspects, the starting buffer comprises about 20.3 mM AMPSO. In certain aspects, the starting buffer comprises about 20.4 mM AMPSO. In certain aspects, the starting buffer comprises about 20.5 mM AMPSO.

In some embodiments, the starting buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, at least about 30 mM, at least about 31 mM, at least about 32 mM, at least about 33 mM, at least about 34 mM, or at least about 35 mM MOPS. In some embodiments, the starting buffer comprises at least about 15 mM MOPS. In some embodiments, the starting buffer comprises at least about 16 mM MOPS. In some embodiments, the starting buffer comprises at least about 17 mM MOPS. In some embodiments, the starting buffer comprises at least about 18 mM MOPS. In some embodiments, the starting buffer comprises at least about 19 mM MOPS. In some embodiments, the starting buffer comprises at least about 20 mM MOPS. In some embodiments, the starting buffer comprises at least about 21 mM MOPS. In some embodiments, the starting buffer comprises at least about 22 mM MOPS.

In some embodiments, the starting buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS. In some embodiments, the starting buffer comprises about 15 mM to about 25 mM MOPS. In some embodiments, the starting buffer comprises about 15 mM to about 20 mM MOPS. In some embodiments, the starting buffer comprises about 16 mM to about 24 mM MOPS. In some embodiments, the starting buffer comprises about 17 mM to about 23 mM MOPS. In some embodiments, the starting buffer comprises about 18 mM to about 22 mM MOPS. In some embodiments, the starting buffer comprises about 19 mM to about 21 mM MOPS. In some embodiments, the starting buffer comprises about 20 mM to about 21 mM MOPS.

In some embodiments, the starting buffer comprises about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 3 mM MOPS. In some embodiments, the starting buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS. In certain embodiments, the starting buffer comprises about 15 mM MOPS. In certain embodiments, the starting buffer comprises about 16 mM MOPS. In certain embodiments, the starting buffer comprises about 17 mM MOPS. In certain embodiments, the starting buffer comprises about 18 mM MOPS. In certain embodiments, the starting buffer comprises about 19 mM MOPS. In certain embodiments, the starting buffer comprises about 19.5 mM MOPS. In certain embodiments, the starting buffer comprises about 20 mM MOPS. In certain embodiments, the starting buffer comprises about 20.5 mM MOPS. In certain embodiments, the starting buffer comprises about 21 mM MOPS. In certain embodiments, the starting buffer comprises about 21.5 mM MOPS. In certain embodiments, the starting buffer comprises about 22 mM MOPS. In certain embodiments, the starting buffer comprises about 23 mM MOPS. In certain embodiments, the starting buffer comprises about 24 mM MOPS. In certain embodiments, the starting buffer comprises about 25 mM MOPS.

In some embodiments, the starting buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10. In certain embodiments, the starting buffer has a pH of about 8.0 to about 10.0, from about 8.1 to about 9.9, from about 8.2 to about 9.8, from about 8.3 to about 9.7, from about 8.4 to about 9.6, from about 8.5 to about 9.5, or from about 9.0 to about 9.5. In certain embodiments, the starting buffer has a pH of about 9.0 to about 9.5. In particular embodiments, the starting buffer has a pH of about 8.5, about 8.6, about 8.7, about 8.8, about 8.9, about 9.0, about 9.1, about 9.2, about 9.3, about 9.4, or about 9.5. In certain embodiments, the starting buffer has a pH of about 9.0. In certain embodiments, the starting buffer has a pH of about 9.1. In certain embodiments, the starting buffer has a pH of about 9.2. In certain embodiments, the starting buffer has a pH of about 9.3. In certain embodiments, the starting buffer has a pH of about 9.4. In certain embodiments, the starting buffer has a pH of about 9.5. In certain aspects, the starting buffer has a pH of about 9.6. In certain aspects, the starting buffer has a pH of about 9.7. In certain aspects, the starting buffer has a pH of about 9.8. In certain aspects, the starting buffer has a pH of about 9.9. In certain aspects, the starting buffer has a pH of about 10.

In particular embodiments, the starting buffer comprises about 9.4 mM AMPSO about 20.5 mM MOPS and a pH of 9.2.

In some aspects, the starting buffer comprises glycine. In certain aspects, the starting buffer comprises at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM glycine.

In some aspects, the starting buffer comprises at least about 5 mM glycine. In some aspects, the starting buffer comprises at least about 6 mM glycine. In some aspects, the starting buffer comprises at least about 7 mM glycine. In some aspects, the starting buffer comprises at least about 8 mM glycine. In some aspects, the starting buffer comprises at least about 9 mM glycine. In some aspects, the starting buffer comprises at least about 9.4 mM glycine. In some aspects, the starting buffer comprises at least about 10 mM glycine. In some aspects, the starting buffer comprises at least about 11 mM glycine. In some aspects, the starting buffer comprises at least about 12 mM glycine. In some aspects, the starting buffer comprises at least about 13 mM glycine. In some aspects, the starting buffer comprises at least about 14 mM glycine. In some aspects, the starting buffer comprises at least about 15 mM glycine. In some aspects, the starting buffer comprises at least about 16 mM glycine. In some aspects, the starting buffer comprises at least about 17 mM glycine. In some aspects, the starting buffer comprises at least about 18 mM glycine. In some aspects, the starting buffer comprises at least about 19 mM glycine. In some aspects, the starting buffer comprises at least about 20 mM glycine.

In certain aspects, the starting buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, about 10 mM to about 15 mM glycine, about 10 mM to about 20 mM glycine, about 10 mM to about 25 mM glycine, about 15 mM to about 20 mM glycine, about 15 mM to about 25 mM glycine, or about 20 mM to about 25 mM glycine.

In some aspects, the starting buffer comprises about 5 mM to about 15 mM glycine. In some aspects, the starting buffer comprises about 5 mM to about 10 mM glycine. In some aspects, the starting buffer comprises about 6 mM to about 10 mM glycine. In some aspects, the starting buffer comprises about 7 mM to about 10 mM glycine. In some aspects, the starting buffer comprises about 8 mM to about 10 mM glycine. In some aspects, the starting buffer comprises about 9 mM to about 10 mM glycine. In some aspects, the starting buffer comprises about 10 mM to about 15 mM glycine. In some aspects, the starting buffer comprises about 10 mM to about 20 mM glycine.

In certain aspects, the starting buffer comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 20.5 mM, about 21 mM, about 22 mM, about 23 mM, about 24 mM, or about 25 glycine. In some aspects, the starting buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM glycine. In certain aspects, the starting buffer comprises about 8 mM glycine. In certain aspects, the starting buffer comprises about 8.5 mM glycine. In certain aspects, the starting buffer comprises about 8.6 mM glycine. In certain aspects, the starting buffer comprises about 8.7 mM glycine. In certain aspects, the starting buffer comprises about 8.8 mM glycine. In certain aspects, the starting buffer comprises about 8.9 mM glycine. In certain aspects, the starting buffer comprises about 9 mM glycine. In certain aspects, the starting buffer comprises about 9.1 mM glycine. In certain aspects, the starting buffer comprises about 9.2 mM glycine. In certain aspects, the starting buffer comprises about 9.3 mM glycine. In certain aspects, the starting buffer comprises about 9.4 mM glycine. In certain aspects, the starting buffer comprises about 9.5 mM glycine. In certain aspects, the starting buffer comprises about 9.6 mM glycine. In certain aspects, the starting buffer comprises about 9.7 mM glycine. In certain aspects, the starting buffer comprises about 9.8 mM glycine. In certain aspects, the starting buffer comprises about 9.9 mM glycine. In certain aspects, the starting buffer comprises about 10.0 mM glycine. In certain aspects, the starting buffer comprises about 10.1 mM glycine. In certain aspects, the starting buffer comprises about 10.2 mM glycine. In certain aspects, the starting buffer comprises about 10.3 mM glycine. In certain aspects, the starting buffer comprises about 10.4 mM glycine. In certain aspects, the starting buffer comprises about 10.5 mM glycine.

In some aspects, the starting buffer comprises about 9.4 mM Glycine and about 20.5 mM AMPSO, at pH 9.8;

In some embodiments, the methods disclosed herein comprise applying a wash buffer to the polishing chromatography matrix. In certain embodiments, the wash buffer is added after the contacting of the mixture comprising the protein product and the contaminant with the polishing chromatography matrix.

One or more column volumes of the wash buffer can be applied to the polishing chromatography matrix. In some embodiments, a single column of the wash buffer is applied after the contacting of the mixture comprising the protein product and the contaminant with the polishing chromatography matrix. In some embodiments, at least 2 column volumes, at least 3 column volumes, at least 4 column volumes, or at least 5 column volumes of the wash buffer are applied to the polishing chromatography matrix. In certain embodiments, each of the one or more column volumes of the wash buffer is applied sequentially, e.g., wherein each subsequent column volume is applied to the column after all or near all of the prior column volume has eluted from the matrix. In some embodiments, each of the one or more column volumes of the wash buffer is applied in tandem, e.g., wherein each subsequent column volume is applied to the column after none of or only a portion of the prior column volume has eluted from the matrix.

In some embodiments, the wash buffer comprises N-(1,1-Dimethyl-2-hydroxyethyl)-3-amino-2-hydroxypropanesulfonic acid (AMPSO). In some embodiments, the wash buffer comprises 3-morpholin-4-ylpropane-1-sulfonic acid (MOPS), or both. In some embodiments, the wash buffer comprises AMPSO and MOPS.

In certain embodiments, the wash buffer comprises at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM AMPSO. In some embodiments, the wash buffer comprises at least about 5 mM AMPSO. In some embodiments, the wash buffer comprises at least about 6 mM AMPSO. In some embodiments, the wash buffer comprises at least about 7 mM AMPSO. In some embodiments, the wash buffer comprises at least about 8 mM AMPSO. In some embodiments, the wash buffer comprises at least about 9 mM AMPSO. In some embodiments, the wash buffer comprises at least about 10 mM AMPSO.

In certain embodiments, the wash buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, or about 10 mM to about 15 mM AMPSO. In some embodiments, the wash buffer comprises about 5 mM to about 15 mM AMPSO. In some embodiments, the wash buffer comprises about 5 mM to about 10 mM AMPSO. In some embodiments, the wash buffer comprises about 6 mM to about 10 mM AMPSO. In some embodiments, the wash buffer comprises about 7 mM to about 10 mM AMPSO. In some embodiments, the wash buffer comprises about 8 mM to about 10 mM AMPSO. In some embodiments, the wash buffer comprises about 9 mM to about 10 mM AMPSO.

In certain embodiments, the wash buffer comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, or about 15 AMPSO. In some embodiments, the wash buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO. In certain embodiments, the wash buffer comprises about 8 mM AMPSO. In certain embodiments, the wash buffer comprises about 8.5 mM AMPSO. In certain embodiments, the wash buffer comprises about 8.6 mM AMPSO. In certain embodiments, the wash buffer comprises about 8.7 mM AMPSO. In certain embodiments, the wash buffer comprises about 8.8 mM AMPSO. In certain embodiments, the wash buffer comprises about 8.9 mM AMPSO. In certain embodiments, the wash buffer comprises about 9 mM AMPSO. In certain embodiments, the wash buffer comprises about 9.1 mM AMPSO. In certain embodiments, the wash buffer comprises about 9.2 mM AMPSO. In certain embodiments, the wash buffer comprises about 9.3 mM AMPSO. In certain embodiments, the wash buffer comprises about 9.4 mM AMPSO. In certain embodiments, the wash buffer comprises about 9.5 mM AMPSO. In certain embodiments, the wash buffer comprises about 9.6 mM AMPSO. In certain embodiments, the wash buffer comprises about 9.7 mM AMPSO. In certain embodiments, the wash buffer comprises about 9.8 mM AMPSO. In certain embodiments, the wash buffer comprises about 9.9 mM AMPSO. In certain embodiments, the wash buffer comprises about 10 mM AMPSO. In certain embodiments, the wash buffer comprises about 10.1 mM AMPSO. In certain embodiments, the wash buffer comprises about 10.2 mM AMPSO. In certain embodiments, the wash buffer comprises about 10.3 mM AMPSO. In certain embodiments, the wash buffer comprises about 10.4 mM AMPSO. In certain embodiments, the wash buffer comprises about 10.5 mM AMPSO.

In some embodiments, the wash buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, at least about 30 mM, at least about 31 mM, at least about 32 mM, at least about 33 mM, at least about 34 mM, or at least about 35 mM MOPS. In some embodiments, the wash buffer comprises at least about 15 mM MOPS. In some embodiments, the wash buffer comprises at least about 16 mM MOPS. In some embodiments, the wash buffer comprises at least about 17 mM MOPS. In some embodiments, the wash buffer comprises at least about 18 mM MOPS. In some embodiments, the wash buffer comprises at least about 19 mM MOPS. In some embodiments, the wash buffer comprises at least about 20 mM MOPS. In some embodiments, the wash buffer comprises at least about 21 mM MOPS. In some embodiments, the wash buffer comprises at least about 22 mM MOPS.

In some embodiments, the wash buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS. In some embodiments, the wash buffer comprises about 15 mM to about 25 mM MOPS. In some embodiments, the wash buffer comprises about 15 mM to about 20 mM MOPS. In some embodiments, the wash buffer comprises about 16 mM to about 24 mM MOPS. In some embodiments, the wash buffer comprises about 17 mM to about 23 mM MOPS. In some embodiments, the wash buffer comprises about 18 mM to about 22 mM MOPS. In some embodiments, the wash buffer comprises about 19 mM to about 21 mM MOPS. In some embodiments, the wash buffer comprises about 20 mM to about 21 mM MOPS.

In some embodiments, the wash buffer comprises about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 3 mM MOPS. In some embodiments, the wash buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS. In certain embodiments, the wash buffer comprises about 15 mM MOPS. In certain embodiments, the wash buffer comprises about 16 mM MOPS. In certain embodiments, the wash buffer comprises about 17 mM MOPS. In certain embodiments, the wash buffer comprises about 18 mM MOPS. In certain embodiments, the wash buffer comprises about 19 mM MOPS. In certain embodiments, the wash buffer comprises about 19.5 mM MOPS. In certain embodiments, the wash buffer comprises about 20 mM MOPS. In certain embodiments, the wash buffer comprises about 20.5 mM MOPS. In certain embodiments, the wash buffer comprises about 21 mM MOPS. In certain embodiments, the wash buffer comprises about 21.5 mM MOPS. In certain embodiments, the wash buffer comprises about 22 mM MOPS. In certain embodiments, the wash buffer comprises about 23 mM MOPS. In certain embodiments, the wash buffer comprises about 24 mM MOPS. In certain embodiments, the wash buffer comprises about 25 mM MOPS.

In some embodiments, the wash buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10. In certain embodiments, the wash buffer has a pH of about 8.0 to about 10.0, from about 8.5 to about 9.5, or from about 9.0 to about 9.5. In certain embodiments, the wash buffer has a pH of about 9.0 to about 9.5. In particular embodiments, the wash buffer has a pH of about 8.5, about 8.6, about 8.7, about 8.8, about 8.9, about 9.0, about 9.1, about 9.2, about 9.3, about 9.4, or about 9.5. In certain embodiments, the wash buffer has a pH of about 9.0. In certain embodiments, the wash buffer has a pH of about 9.1. In certain embodiments, the wash buffer has a pH of about 9.2. In certain embodiments, the wash buffer has a pH of about 9.3. In certain embodiments, the wash buffer has a pH of about 9.4. In certain embodiments, the wash buffer has a pH of about 9.5.

In particular embodiments, the wash buffer comprises about 9.4 mM AMPSO about 20.5 mM MOPS and a pH of 9.2. In certain embodiments, the wash buffer and the starting buffer are the same. In some embodiments, the wash buffer and the starting buffer are different.

In certain embodiments, the methods described herein further comprise applying an elution buffer to the polishing chromatography matrix. In some embodiments, the elution buffer is applied immediately after the contacting of the mixture comprising the protein product and the contaminant with the polishing chromatography matrix, e.g., without applying a wash buffer. In some embodiments, the elution buffer is applied after applying one or more volumes of the wash buffer to the polishing chromatography matrix.

In some embodiments, the elution buffer comprises MOPS. In some embodiments, the elution buffer comprises 4-Morpholineethanesulfonic acid monohydrate (MES). In some embodiments, the elution buffer comprises MOPS and MES.

In some embodiments, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM MOPS. In certain embodiments, the elution buffer comprises at least about 7 mM MOPS. In certain embodiments, the elution buffer comprises at least about 8 mM MOPS. In certain embodiments, the elution buffer comprises at least about 9 mM MOPS. In certain embodiments, the elution buffer comprises at least about 10 mM MOPS. In certain embodiments, the elution buffer comprises at least about 11 mM MOPS. In certain embodiments, the elution buffer comprises at least about 12 mM MOPS.

In some embodiments, the elution buffer comprises about 0.1 mM to about 20 mM, about 0.5 mM to about 15 mM, about 5 mM to about 15 mM, about 6 mM to about 15 mM, about 7 mM to about 15 mM, about 8 mM to about 15 mM, about 9 mM to about 15 mM, about 10 mM to about 15 mM, about 10 mM to about 14 mM, or about 11 mM to about 13 mM MOPS. In certain embodiments, the elution buffer comprises about 5 mM to about 15 mM MOPS. In some embodiments, the elution buffer comprises about 10 mM to 15 mM MOPS. In some embodiments, the elution buffer comprises about 8 mM to 15 mM MOPS. In some embodiments, the elution buffer comprises about 9 mM to 14 mM MOPS. In some embodiments, the elution buffer comprises about 10 mM to 13 mM MOPS. In some embodiments, the elution buffer comprises about 11 mM to 13 mM MOPS. In some embodiments, the elution buffer comprises about 11 mM to 12 mM MOPS. In some embodiments, the elution buffer comprises about 12 mM to 13 mM MOPS.

In some embodiments, the elution buffer comprises about 5 mM, about 6 mM, about 7 mM, about 8 mM, about 9 mM, about 10 mM, about 11 mM, about 12 mM, about 13 mM, about 14 mM, or about 15 mM MOPS. In some embodiments, the elution buffer comprises about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, or about 13.0 mM MOPS. In certain embodiments, the elution buffer comprises about 10 mM MOPS. In certain embodiments, the elution buffer comprises about 10.5 mM MOPS. In certain embodiments, the elution buffer comprises about 11 mM MOPS. In certain embodiments, the elution buffer comprises about 11.5 mM MOPS. In certain embodiments, the elution buffer comprises about 12 mM MOPS. In certain embodiments, the elution buffer comprises about 12.5 mM MOPS. In certain embodiments, the elution buffer comprises about 13 mM MOPS. In certain embodiments, the elution buffer comprises about 13.5 mM MOPS. In certain embodiments, the elution buffer comprises about 14 mM MOPS.

In some aspects, the elution buffer comprises AMPSO. In some aspects, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM AMPSO. In certain aspects, the elution buffer comprises at least about 7 mM AMPSO. In certain aspects, the elution buffer comprises at least about 8 mM AMPSO. In certain aspects, the elution buffer comprises at least about 9 mM AMPSO. In certain aspects, the elution buffer comprises at least about 10 mM AMPSO. In certain aspects, the elution buffer comprises at least about 11 mM AMPSO. In certain aspects, the elution buffer comprises at least about 12 mM AMPSO. In certain aspects, the elution buffer comprises at least about 13 mM AMPSO. In certain aspects, the elution buffer comprises at least about 14 mM AMPSO. In certain aspects, the elution buffer comprises at least about 15 mM AMPSO.

In some aspects, the elution buffer comprises about 0.1 mM to about 20 mM, about 0.5 mM to about 15 mM, about 5 mM to about 15 mM, about 6 mM to about 15 mM, about 7 mM to about 15 mM, about 8 mM to about 15 mM, about 9 mM to about 15 mM, about 10 mM to about 15 mM, about 10 mM to about 14 mM, or about 11 mM to about 13 mM AMPSO. In certain aspects, the elution buffer comprises about 5 mM to about 15 mM AMPSO. In some aspects, the elution buffer comprises about 10 mM to 15 mM AMPSO. In some aspects, the elution buffer comprises about 8 mM to 15 mM AMPSO. In some aspects, the elution buffer comprises about 9 mM to 14 mM AMPSO. In some aspects, the elution buffer comprises about 10 mM to 13 mM AMPSO. In some aspects, the elution buffer comprises about 11 mM to 13 mM AMPSO. In some aspects, the elution buffer comprises about 11 mM to 12 mM AMPSO. In some aspects, the elution buffer comprises about 12 mM to 13 mM AMPSO.

In some aspects, the elution buffer comprises about 5 mM, about 6 mM, about 7 mM, about 8 mM, about 9 mM, about 10 mM, about 11 mM, about 12 mM, about 13 mM, about 14 mM, or about 15 mM AMPSO. In some aspects, the elution buffer comprises about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, or about 13.0 mM AMPSO. In certain aspects, the elution buffer comprises about 10 mM AMPSO. In certain aspects, the elution buffer comprises about 10.5 mM AMPSO. In certain aspects, the elution buffer comprises about 11 mM AMPSO. In certain aspects, the elution buffer comprises about 11.5 mM AMPSO. In certain aspects, the elution buffer comprises about 12 mM AMPSO. In certain aspects, the elution buffer comprises about 12.5 mM AMPSO. In certain aspects, the elution buffer comprises about 13 mM AMPSO. In certain aspects, the elution buffer comprises about 13.5 mM AMPSO. In certain aspects, the elution buffer comprises about 14 mM AMPSO.

In some embodiments, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM MES. In certain embodiments, the elution buffer comprises at least about 5 mM MES. In certain embodiments, the elution buffer comprises at least about 6 mM MES. In certain embodiments, the elution buffer comprises at least about 7 mM MES. In certain embodiments, the elution buffer comprises at least about 8 mM MES. In certain embodiments, the elution buffer comprises at least about 9 mM MES. In certain embodiments, the elution buffer comprises at least about 10 mM MES. In certain embodiments, the elution buffer comprises at least about 11 mM MES. In certain embodiments, the elution buffer comprises at least about 12 mM MES. In certain embodiments, the elution buffer comprises at least about 13 mM MES. In certain embodiments, the elution buffer comprises at least about 14 mM MES. In certain embodiments, the elution buffer comprises at least about 15 mM MES.

In some embodiments, the elution buffer comprises about 0.1 mM to about 20 mM, about 0.5 mM to about 15 mM, about 5 mM to about 15 mM, about 6 mM to about 14 mM, about 7 mM to about 13 mM, about 8 mM to about 12 mM, about 9 mM to about 11 mM, about 5 mM to about 10 mM, or about 10 mM to about 15 mM MES. In some embodiments, the elution buffer comprises about 5 mM to about 15 mM MES. In some embodiments, the elution buffer comprises about 5 mM to about 10 mM MES. In some embodiments, the elution buffer comprises about 10 mM to about 15 mM MES. In some embodiments, the elution buffer comprises about 6 mM to about 14 mM MES. In some embodiments, the elution buffer comprises about 7 mM to about 13 mM MES. In some embodiments, the elution buffer comprises about 8 mM to about 12 mM MES. In some embodiments, the elution buffer comprises about 9 mM to about 11 mM MES.

In some embodiments, the elution buffer comprises about 5 mM, about 6 mM, about 7 mM, about 8 mM, about 9 mM, about 10 mM, about 11 mM, about 12 mM, about 13 mM, about 14 mM, or about 15 mM MES. In some embodiments, the elution buffer comprises about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10.0 mM, about 10.1 mM, about 10.2 mM, about 10.3 mM, about 10.4 mM, about 10.5 mM, about 10.6 mM, about 10.7 mM, about 10.8 mM, about 10.9 mM, or about 11.0 mM MES. In certain embodiments, the elution buffer comprises about 7 mM MES. In some embodiments, the elution buffer comprises about 7.5 mM MES. In some embodiments, the elution buffer comprises about 8.0 mM MES. In some embodiments, the elution buffer comprises about 8.5 mM MES. In some embodiments, the elution buffer comprises about 9.0 mM MES. In some embodiments, the elution buffer comprises about 9.5 mM MES. In some embodiments, the elution buffer comprises about 10.0 mM MES. In some embodiments, the elution buffer comprises about 10.5 mM MES. In some embodiments, the elution buffer comprises about 11.0 mM MES. In some embodiments, the elution buffer comprises about 11.5 mM MES. In some embodiments, the elution buffer comprises about 12.0 mM MES. In some embodiments, the elution buffer comprises about 12.5 mM MES. In some embodiments, the elution buffer comprises about 13.0 mM MES.

In some aspects, the elution buffer comprises tricine. In some aspects, the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM tricine. In certain aspects, the elution buffer comprises at least about 7 mM tricine. In certain aspects, the elution buffer comprises at least about 8 mM tricine. In certain aspects, the elution buffer comprises at least about 9 mM tricine. In certain aspects, the elution buffer comprises at least about 10 mM tricine. In certain aspects, the elution buffer comprises at least about 11 mM tricine. In certain aspects, the elution buffer comprises at least about 12 mM tricine. In certain aspects, the elution buffer comprises at least about 13 mM tricine. In certain aspects, the elution buffer comprises at least about 14 mM tricine. In certain aspects, the elution buffer comprises at least about 15 mM tricine.

In some aspects, the elution buffer comprises about 0.1 mM to about 20 mM, about 0.5 mM to about 15 mM, about 5 mM to about 15 mM, about 6 mM to about 15 mM, about 7 mM to about 15 mM, about 8 mM to about 15 mM, about 9 mM to about 15 mM, about 10 mM to about 15 mM, about 10 mM to about 14 mM, or about 11 mM to about 13 mM tricine. In certain aspects, the elution buffer comprises about 5 mM to about 15 mM tricine. In some aspects, the elution buffer comprises about 10 mM to 15 mM tricine. In some aspects, the elution buffer comprises about 8 mM to 15 mM tricine. In some aspects, the elution buffer comprises about 9 mM to 14 mM tricine. In some aspects, the elution buffer comprises about 10 mM to 13 mM tricine. In some aspects, the elution buffer comprises about 11 mM to 13 mM tricine. In some aspects, the elution buffer comprises about 11 mM to 12 mM tricine. In some aspects, the elution buffer comprises about 12 mM to 13 mM tricine.

In some aspects, the elution buffer comprises about 5 mM, about 6 mM, about 7 mM, about 8 mM, about 9 mM, about 10 mM, about 11 mM, about 12 mM, about 13 mM, about 14 mM, or about 15 mM tricine. In some aspects, the elution buffer comprises about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, or about 13.0 mM tricine. In certain aspects, the elution buffer comprises about 10 mM tricine. In certain aspects, the elution buffer comprises about 10.5 mM tricine. In certain aspects, the elution buffer comprises about 11 mM tricine. In certain aspects, the elution buffer comprises about 11.5 mM tricine. In certain aspects, the elution buffer comprises about 12 mM tricine. In certain aspects, the elution buffer comprises about 12.5 mM tricine. In certain aspects, the elution buffer comprises about 13 mM tricine. In certain aspects, the elution buffer comprises about 13.5 mM tricine. In certain aspects, the elution buffer comprises about 14 mM tricine.

In some embodiments, the pH of the elution buffer is different from the pH of the starting buffer and/or the pH of the wash buffer. In certain embodiments, the pH of the elution buffer is lower than the pH of the starting buffer and/or the pH of the wash buffer. The pH of the elution buffer can depend on the apparent isoelectric point of the protein product. The apparent isoelectric point differs somewhat from the actual isoelectric point of the protein since the former property depends partly on the column packing properties and other related physical parameters.

In some embodiments, the elution buffer has a pH of less than about 9.0, less than about 8.9, less than about 8.8, less than about 8.7, less than about 8.6, less than about 8.5, less than about 8.4, less than about 8.3, less than about 8.2, less than about 8.1, less than about 8.0, less than about 7.9, less than about 7.8, less than about 7.7, less than about 7.6, less than about 7.5, less than about 7.4, less than about 7.3, less than about 7.2, less than about 7.1, less than about 7.0, less than about 6.9, less than about 6.8, less than about 6.7, less than about 6.6, less than about 6.5, less than about 6.4, less than about 6.3, less than about 6.2, less than about 6.1, less than about 6.0, less than about 5.9, less than about 5.8, less than about 5.7, less than about 5.6, less than about 5.5, less than about 5.4, less than about 5.3, less than about 5.2, or less than about 5.1. In certain aspects, the elution buffer has a pH of less than about 8.1. In certain embodiments, the elution buffer has a pH of less than about 8.0. In certain embodiments, the elution buffer has a pH of less than about 7.5. In certain embodiments, the elution buffer has a pH of less than about 7.0. In certain embodiments, the elution buffer has a pH of less than about 6.5. In certain embodiments, the elution buffer has a pH of less than about 6.0. In some aspects, the elution buffer has a pH of about 8.1. In some aspects, the elution buffer has a pH of about 8.07. In some embodiments, the elution buffer has a pH of about 6.5. In some embodiments, the elution buffer has a pH of about 6.4. In some embodiments, the elution buffer has a pH of about 6.3. In some embodiments, the elution buffer has a pH of about 6.2. In some embodiments, the elution buffer has a pH of about 6.1. In some embodiments, the elution buffer has a pH of about 6.0.

In some embodiments, the elution buffer comprises about 12 mM MOPS and about 10 mM MES and a pH of about 6.2.

In some aspects, the elution buffer comprises about 12 mM AMPSO and about 10 mM tricine and a pH of about 8.07.

I.B. Retained Gradient

In certain embodiments, the polishing chromatography matrix disclosed herein is used together with a retained gradient. In some embodiments, the retained gradient comprises a retained pH gradient. In some embodiments, the retained gradient comprises a retained ionic strength gradient. In some embodiments, the retained gradient comprises a retained pH gradient and a retained ionic strength gradient.

In certain embodiments, the retained gradient is applied to the polishing chromatography matrix before the contacting of the mixture comprising the protein product and the contaminant with the polishing chromatography matrix. In some embodiments, the retained gradient is applied to the polishing chromatography matrix after the contacting of the mixture comprising the protein product and the contaminant with the polishing chromatography matrix.

In certain embodiments, the starting buffer comprises more than one buffering species, each of the more than one buffering species travels through the matrix at a different velocity.

In certain embodiments, the retained gradient is applied to the polishing chromatography matrix during a wash. In certain embodiments, the wash buffer comprises more than one buffering species, wherein the more than one buffering species flow through the matrix at different velocities, thereby establishing a retained gradient.

In certain embodiments, the retained gradient is created by performing a stepwise change between the starting buffer or the wash buffer and the elution buffer. In some embodiments, the stepwise change between the starting buffer or the wash buffer and the elution buffer is performed by applying serial dilutions of the elution buffer in the starting buffer or the wash buffer to the polishing chromatography matrix.

In certain embodiments, the elution buffer comprises more than one buffering species, wherein the more than one buffering species flow through the matrix at different velocities, thereby establishing a retained gradient.

In some embodiments, the more than one buffering species are selected from acetic acid, Tris(hydroxymethyl)aminomethane (Tris), 2-[Bis(2-hydroxyethyl)amino]-2-(hydroxymethyl)propane-1,3-diol (Bis-Tris), 2-(N-morpholino)ethanesulfonic acid (MES), N-tris(hydroxymethyl)methyl-3-aminopropanesulfonic acid (TAPS), 3-(N-morpholino)propanesulfonic acid (MOPS), N-(1,1-dimethyl-2-hydroxyethyl)-3-amino-2-hydroxypropanesulfonic acid (AMPSO), N-(2-Hydroxy-1,1-bis(hydroxymethyl)ethyl) (tricine), glycine, histidine, and any combination thereof.

In some embodiments, the more than one buffering species comprises acetic acid. In some embodiments, the more than one buffering species comprises acetic acid and one or more additional buffering species selected from Tris, Bis-Tris, MES, TAPS, MOPS, AMPSO, tricine, glycine, histidine, and any combination thereof. In some embodiments, the more than one buffering species comprises acetic acid and Tris. In some embodiments, the more than one buffering species comprises acetic acid and Bis-Tris. In some embodiments, the more than one buffering species comprises acetic acid and MES. In some embodiments, the more than one buffering species comprises acetic acid and TAPS. In some embodiments, the more than one buffering species comprises acetic acid and MOPS. In some embodiments, the more than one buffering species comprises acetic acid and AMPSO. In some embodiments, the more than one buffering species comprises acetic acid and tricine. In some embodiments, the more than one buffering species comprises acetic acid and glycine. In some embodiments, the more than one buffering species comprises acetic acid and histidine.

In some embodiments, the more than one buffering species comprises Tris. In some embodiments, the more than one buffering species comprises Tris and one or more additional buffering species selected from acetic acid, Bis-Tris, MES, TAPS, MOPS, AMPSO, tricine, glycine, histidine, and any combination thereof. In some embodiments, the more than one buffering species comprises Tris and Bis-Tris. In some embodiments, the more than one buffering species comprises Tris and MES. In some embodiments, the more than one buffering species comprises Tris and TAPS. In some embodiments, the more than one buffering species comprises Tris and MOPS. In some embodiments, the more than one buffering species comprises Tris and AMPSO. In some embodiments, the more than one buffering species comprises Tris and tricine. In some embodiments, the more than one buffering species comprises Tris and glycine. In some embodiments, the more than one buffering species comprises Tris and histidine.

In some embodiments, the more than one buffering species comprises Bis-Tris. In some embodiments, the more than one buffering species comprises Bis-Tris and one or more additional buffering species selected from acetic acid, Tris, MES, TAPS, MOPS, AMPSO, tricine, glycine, histidine, and any combination thereof. In some embodiments, the more than one buffering species comprises Bis-Tris and MES. In some embodiments, the more than one buffering species comprises Bis-Tris and TAPS. In some embodiments, the more than one buffering species comprises Bis-Tris and MOPS. In some embodiments, the more than one buffering species comprises Bis-Tris and AMPSO. In some embodiments, the more than one buffering species comprises Bis-Tris and tricine. In some embodiments, the more than one buffering species comprises Bis-Tris and glycine. In some embodiments, the more than one buffering species comprises Bis-Tris and histidine.

In some embodiments, the more than one buffering species comprises MES. In some embodiments, the more than one buffering species comprises MES and one or more additional buffering species selected from acetic acid, Tris, Bis-Tris, TAPS, MOPS, AMPSO, tricine, glycine, histidine, and any combination thereof. In some embodiments, the more than one buffering species comprises MES and TAPS. In some embodiments, the more than one buffering species comprises MES and MOPS. In some embodiments, the more than one buffering species comprises MES and AMPSO. In some embodiments, the more than one buffering species comprises MES and tricine. In some embodiments, the more than one buffering species comprises MES and glycine. In some embodiments, the more than one buffering species comprises MES and histidine.

In some embodiments, the more than one buffering species comprises TAPS. In some embodiments, the more than one buffering species comprises TAPS and one or more additional buffering species selected from acetic acid, Tris, Bis-Tris, MES, MOPS, AMPSO, tricine, glycine, histidine, and any combination thereof. In some embodiments, the more than one buffering species comprises TAPS and MOPS. In some embodiments, the more than one buffering species comprises TAPS and AMPSO. In some embodiments, the more than one buffering species comprises TAPS and tricine. In some embodiments, the more than one buffering species comprises TAPS and glycine. In some embodiments, the more than one buffering species comprises TAPS and histidine.

In some embodiments, the more than one buffering species comprises MOPS. In some embodiments, the more than one buffering species comprises MOPS and one or more additional buffering species selected from acetic acid, Tris, Bis-Tris, MES, TAPS, AMPSO, tricine, glycine, histidine, and any combination thereof. In some embodiments, the more than one buffering species comprises MOPS and AMPSO. In some embodiments, the more than one buffering species comprises MOPS and tricine. In some embodiments, the more than one buffering species comprises MOPS and glycine. In some embodiments, the more than one buffering species comprises MOPS and histidine.

In some embodiments, the more than one buffering species comprises AMPSO. In some embodiments, the more than one buffering species comprises AMPSO and one or more additional buffering species selected from acetic acid, Tris, Bis-Tris, MES, TAPS, MOPS, tricine, glycine, histidine, and any combination thereof. In some embodiments, the more than one buffering species comprises AMPSO and tricine. In some embodiments, the more than one buffering species comprises AMPSO and glycine. In some embodiments, the more than one buffering species comprises AMPSO and histidine.

In some embodiments, the more than one buffering species comprises tricine. In some embodiments, the more than one buffering species comprises tricine and one or more additional buffering species selected from acetic acid, Tris, Bis-Tris, MES, TAPS, MOPS, AMPSO, glycine, histidine, and any combination thereof. In some embodiments, the more than one buffering species comprises tricine and glycine. In some embodiments, the more than one buffering species comprises tricine and histidine.

In some embodiments, the more than one buffering species comprises glycine. In some embodiments, the more than one buffering species comprises glycine and one or more additional buffering species selected from acetic acid, Tris, Bis-Tris, MES, TAPS, MOPS, AMPSO, tricine, histidine, and any combination thereof. In some embodiments, the more than one buffering species comprises glycine and histidine.

In some embodiments, the more than one buffering species comprises histidine. In some embodiments, the more than one buffering species comprises histidine and one or more additional buffering species selected from acetic acid, Tris, Bis-Tris, MES, TAPS, MOPS, AMPSO, tricine, glycine, and any combination thereof.

In some embodiments, the more than one buffering species in the starting buffer and/or the wash buffer are the same as the more than one buffering species in the elution buffer. In some embodiments, the starting buffer and/or the wash buffer comprises at least one buffering species that is not present in the elution buffer. In some embodiments, the elution buffer comprises at least one buffering species that is present in the starting buffer and/or the wash buffer. In certain embodiments, the elution buffer comprises at least one buffering species that is present in the starting buffer and/or the wash buffer, wherein the at least one buffering species is present in the elution buffer at a concentration that is different from the concentration of the at least one buffering species in the starting buffer and/or the elution buffer.

In certain embodiments, the retained gradient comprises a retained pH gradient. The retained pH gradient can be determined based on the apparent isoelectric points of the protein product and the contaminant. In some embodiments, a 2D analytical fractionation process development tool is used to determine the apparent isoelectric point of the protein product and/or the contaminant. This tool determines the quantity of both the impurities and protein product, e.g., mAb product, in a mixture, e.g., a protein A pool, as well as the “apparent” isoelectric points of these species that pertain to the methods described herein. In certain embodiments, a specific anion-exchange column packing and a generic pH gradient consisting of a number X of small self-sharpening pH fronts is used as a first dimension of the 2D method. In certain embodiments, the number X is selected from at least about 5, at least about 6, at least about 7, at least about 8, at least about 9, at least about 10, at least about 11, at least about 12, at least about 13, at least about 14, and at least about 15. In certain embodiments, the number X is about 10. In some embodiments, column effluent fractions from the first dimension are then collected from each of the small self-sharpening pH fronts. In some embodiments, each of the column effluent fractions is then subjected to high sensitivity silver-stained SDS-PAGE to form a 2D map of the components of the mixture, e.g., a protein A pool. By comparing the relative positions of the gel bands for the host cell proteins (HCPs) and antibody product components, the pH gradient can be selected that applies to the specific column packing, mixture, and purification objective under consideration. The starting and elution buffers corresponding to the selected pH gradient can then be designed using software based on local-equilibrium theory.

In some embodiments, the starting buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min.

In some embodiments, the starting buffer is applied to the polishing chromatography matrix at a volumetric flow rate corresponding to a superficial liquid velocity of about 0.1 cm/min to about 10.0 cm/min, about 0.5 cm/min to about 10.0 cm/min, about 1.0 cm/min to about 10.0 cm/min, about 1.0 cm/min to about 9.0 cm/min, about 1.0 cm/min to about 8.0 cm/min, about 1.0 cm/min to about 7.0 cm/min, about 1.0 cm/min to about 6.0 cm/min, about 1.0 cm/min to about 5.0 cm/min, about 1.0 cm/min to about 4.0 cm/min, about 1.0 cm/min to about 3.0 cm/min, about 1 cm/min to about 2.0 cm/min, about 1.0 cm/min to about 10.0 cm/min, about 2.0 cm/min to about 10.0 cm/min, about 3.0 cm/min to about 10.0 cm/min, about 4.0 cm/min to about 10.0 cm/min, about 5.0 cm/min to about 10.0 cm/min, about 6.0 cm/min to about 10.0 cm/min, about 17 cm/min to about 10.0 cm/min, about 8.0 cm/min to about 10.0 cm/min, or about 9.0 cm/min to about 10.0 cm/min. In certain embodiments, the starting buffer is applied to the polishing chromatography matrix at a volumetric flow rate corresponding to a superficial liquid velocity of about 1.0 cm/min to about 10.0 cm/min. In certain embodiments, the starting buffer is applied to the polishing chromatography matrix at a volumetric flow rate corresponding to a superficial liquid velocity of about 2.5 cm/min to about 10.0 cm/min. In certain embodiments, the starting buffer is applied to the polishing chromatography matrix at a volumetric flow rate corresponding to a superficial liquid velocity of about 1.0 cm/min to about 5.0 cm/min. In certain embodiments, the starting buffer is applied to the polishing chromatography matrix at a volumetric flow rate corresponding to a superficial liquid velocity of about 5.0 cm/min to about 10.0 cm/min.

In some embodiments, the starting buffer is applied to the polishing chromatography matrix at a volumetric flow rate corresponding to a superficial liquid velocity of about 2.8 cm/min, about 2.9 cm/min, about 3.0 cm/min, about 3.1 cm/min, about 3.2 cm/min, about 3.3 cm/min, about 3.3 cm/min, about 3.4 cm/min, about 3.5 cm/min, about 3.6 cm/min, about 3.7 cm/min, about 3.8 cm/min, about 3.9 cm/min, about 4.0 cm/min, about 4.1 cm/min, about 4.2 cm/min, about 4.3 cm/min, about 4.4 cm/min, about 4.5 cm/min, about 4.6 cm/min, about 4.7 cm/min, about 4.8 cm/min, about 4.9 cm/min, or about 5.0 cm/min. In certain embodiments, the starting buffer is applied to the polishing chromatography matrix at a volumetric flow rate corresponding to a superficial liquid velocity of about 2.5 cm/min. In certain embodiments, the starting buffer is applied to the polishing chromatography matrix at a volumetric flow rate corresponding to a superficial liquid velocity of about 3.0 cm/min. In certain embodiments, the starting buffer is applied to the polishing chromatography matrix at a volumetric flow rate corresponding to a superficial liquid velocity of about 3.5 cm/min. In certain embodiments, the starting buffer is applied to the polishing chromatography matrix at a volumetric flow rate corresponding to a superficial liquid velocity of about 4.0 cm/min. In certain embodiments, the starting buffer is applied to the polishing chromatography matrix at a volumetric flow rate corresponding to a superficial liquid velocity of about 4.5 cm/min. In certain embodiments, the starting buffer is applied to the polishing chromatography matrix at a volumetric flow rate corresponding to a superficial liquid velocity of about 5.0 cm/min. In certain embodiments, the starting buffer is applied to the polishing chromatography matrix at a volumetric flow rate corresponding to a superficial liquid velocity of about 6.0 cm/min. In certain embodiments, the starting buffer is applied to the polishing chromatography matrix at a volumetric flow rate corresponding to a superficial liquid velocity of about 7.0 cm/min. In certain embodiments, the starting buffer is applied to the polishing chromatography matrix at a volumetric flow rate corresponding to a superficial liquid velocity of about 8.0 cm/min. In certain embodiments, the starting buffer is applied to the polishing chromatography matrix at a volumetric flow rate corresponding to a superficial liquid velocity of about 9.0 cm/min. In certain embodiments, the starting buffer is applied to the polishing chromatography matrix at a volumetric flow rate corresponding to a superficial liquid velocity of about 10.0 cm/min.

In some embodiments, the wash buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min.

In some embodiments, the wash buffer is applied to the polishing chromatography matrix at a volumetric flow rate corresponding to a superficial liquid velocity of about 0.1 cm/min to about 10.0 cm/min, about 0.5 cm/min to about 10.0 cm/min, about 1.0 cm/min to about 10.0 cm/min, about 1.0 cm/min to about 9.0 cm/min, about 1.0 cm/min to about 8.0 cm/min, about 1.0 cm/min to about 7.0 cm/min, about 1.0 cm/min to about 6.0 cm/min, about 1.0 cm/min to about 5.0 cm/min, about 1.0 cm/min to about 4.0 cm/min, about 1.0 cm/min to about 3.0 cm/min, about 1 cm/min to about 2.0 cm/min, about 1.0 cm/min to about 10.0 cm/min, about 2.0 cm/min to about 10.0 cm/min, about 3.0 cm/min to about 10.0 cm/min, about 4.0 cm/min to about 10.0 cm/min, about 5.0 cm/min to about 10.0 cm/min, about 6.0 cm/min to about 10.0 cm/min, about 17 cm/min to about 10.0 cm/min, about 8.0 cm/min to about 10.0 cm/min, or about 9.0 cm/min to about 10.0 cm/min. In certain embodiments, the wash buffer is applied to the polishing chromatography matrix at a volumetric flow rate corresponding to a superficial liquid velocity of about 1.0 cm/min to about 10.0 cm/min. In certain embodiments, the wash buffer is applied to the polishing chromatography matrix at a volumetric flow rate corresponding to a superficial liquid velocity of about 1.0 cm/min to about 5.0 cm/min. In certain embodiments, the wash buffer is applied to the polishing chromatography matrix at a volumetric flow rate corresponding to a superficial liquid velocity of about 2.5 cm/min to about 10.0 cm/min. In certain embodiments, the wash buffer is applied to the polishing chromatography matrix at a volumetric flow rate corresponding to a superficial liquid velocity of about 5.0 cm/min to about 10.0 cm/min.

In some embodiments, the wash buffer is applied to the polishing chromatography matrix at a volumetric flow rate corresponding to a superficial liquid velocity of about 2.8 cm/min, about 2.9 cm/min, about 3.0 cm/min, about 3.1 cm/min, about 3.2 cm/min, about 3.3 cm/min, about 3.3 cm/min, about 3.4 cm/min, about 3.5 cm/min, about 3.6 cm/min, about 3.7 cm/min, about 3.8 mL/min, about 3.9 mL/min, about 4.0 cm/min, about 4.1 mL/min, about 4.2 cm/min, about 4.3 cm/min, about 4.4 mL/min, about 4.5 cm/min, about 4.6 cm/min, about 4.7 cm/min, about 4.8 cm/min, about 4.9 cm/min, or about 5.0 cm/min. In certain embodiments, the wash buffer is applied to the polishing chromatography matrix at a volumetric flow rate corresponding to a superficial liquid velocity of about 2.5 cm/min. In certain embodiments, the wash buffer is applied to the polishing chromatography matrix at a volumetric flow rate corresponding to a superficial liquid velocity of about 3.0 cm/min. In certain embodiments, the wash buffer is applied to the polishing chromatography matrix at a volumetric flow rate corresponding to a superficial liquid velocity of about 3.5 cm/min. In certain embodiments, the wash buffer is applied to the polishing chromatography matrix at a volumetric flow rate corresponding to a superficial liquid velocity of about 4.0 cm/min. In certain embodiments, the wash buffer is applied to the polishing chromatography matrix at a volumetric flow rate corresponding to a superficial liquid velocity of about 4.5 cm/min. In certain embodiments, the wash buffer is applied to the polishing chromatography matrix at a volumetric flow rate corresponding to a superficial liquid velocity of about 5.0 cm/min. In certain embodiments, the wash buffer is applied to the polishing chromatography matrix at a volumetric flow rate corresponding to a superficial liquid velocity of about 6.0 cm/min. In certain embodiments, the wash buffer is applied to the polishing chromatography matrix at a volumetric flow rate corresponding to a superficial liquid velocity of about 7.0 cm/min. In certain embodiments, the wash buffer is applied to the polishing chromatography matrix at a volumetric flow rate corresponding to a superficial liquid velocity of about 8.0 cm/min. In certain embodiments, the wash buffer is applied to the polishing chromatography matrix at a volumetric flow rate corresponding to a superficial liquid velocity of about 9.0 cm/min. In certain embodiments, the wash buffer is applied to the polishing chromatography matrix at a volumetric flow rate corresponding to a superficial liquid velocity of about 10.0 cm/min.

In some embodiments, the stepwise change between the starting buffer and the elution buffer is applied to the polishing chromatography matrix at a volumetric flow rate corresponding to a superficial liquid velocity of at least about 1.4 cm/min, at least about 1.5 cm/min, at least about 1.6 cm/min, at least about 1.7 cm/min, at least about 1.8 cm/min, at least about 1.9 cm/min, at least about 2.0 cm/min, at least about 2.1 cm/min, at least about 2.2 cm/min, at least about 2.3 cm/min, at least about 2.4 cm/min, at least about 2.5 cm/min, at least about 2.6 cm/min, at least about 2.7 cm/min, at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min or at least about 4.0 cm/min.

In some embodiments, the stepwise change between the starting buffer and the elution buffer is applied to the polishing chromatography matrix at a volumetric flow rate corresponding to a superficial liquid velocity of about 0.1 cm/min to about 10.0 cm/min, about 0.5 cm/min to about 10.0 cm/min, about 1.0 cm/min to about 10.0 cm/min, about 1.0 cm/min to about 9.0 cm/min, about 1.0 cm/min to about 8.0 cm/min, about 1.0 cm/min to about 7.0 cm/min, about 1.0 cm/min to about 6.0 cm/min, about 1.0 cm/min to about 5.0 cm/min, about 1.0 cm/min to about 4.0 cm/min, about 1.0 cm/min to about 3.0 cm/min, about 1 cm/min to about 2.0 cm/min, about 1.0 cm/min to about 10.0 cm/min, about 2.0 cm/min to about 10.0 cm/min, about 3.0 cm/min to about 10.0 cm/min, about 4.0 cm/min to about 10.0 cm/min, about 5.0 cm/min to about 10.0 cm/min, about 6.0 cm/min to about 10.0 cm/min, about 17 cm/min to about 10.0 cm/min, about 8.0 cm/min to about 10.0 cm/min, or about 9.0 cm/min to about 10.0 cm/min. In certain embodiments, the stepwise change between the starting buffer and the elution buffer is applied to the polishing chromatography matrix at a volumetric flow rate corresponding to a superficial liquid velocity of about 1.0 cm/min to about 10.0 cm/min. In certain embodiments, the stepwise change between the starting buffer and the elution buffer is applied to the polishing chromatography matrix at a volumetric flow rate corresponding to a superficial liquid velocity of about 2.5 cm/min to about 10.0 cm/min. In certain embodiments, the stepwise change between the starting buffer and the elution buffer is applied to the polishing chromatography matrix at a volumetric flow rate corresponding to a superficial liquid velocity of about 1.0 cm/min to about 5.0 cm/min. In certain embodiments, the stepwise change between the starting buffer and the elution buffer is applied to the polishing chromatography matrix at a volumetric flow rate corresponding to a superficial liquid velocity of about 5.0 cm/min to about 10.0 cm/min.

In some embodiments, the stepwise change between the starting buffer and the elution buffer is applied to the polishing chromatography matrix at a volumetric flow rate corresponding to a superficial liquid velocity of about 1.4 cm/min, about 1.5 cm/min, about 1.6 cm/min, about 1.7 cm/min, about 1.8 cm/min, about 1.9 cm/min, about 2.0 cm/min, about 2.1 cm/min, about 2.2 cm/min, about 2.3 cm/min, about 2.4 cm/min, about 2.5 mL/min, about 2.6 mL/min, about 2.7 cm/min, about 2.8 mL/min, about 2.9 cm/min, about 3.0 cm/min, about 3.1 mL/min, about 3.2 cm/min, about 3.3 cm/min, about 3.4 cm/min, about 3.5 cm/min, about 3.6 cm/min, about 3.7 cm/min, about 3.8 cm/min, about 3.9 cm/min or about 4.0 cm/min. In certain embodiments, the stepwise change between the starting buffer and the elution buffer is applied to the polishing chromatography matrix at a volumetric flow rate corresponding to a superficial liquid velocity of about 1.0 cm/min. In certain embodiments, the stepwise change between the starting buffer and the elution buffer is applied to the polishing chromatography matrix at a volumetric flow rate corresponding to a superficial liquid velocity of about 1.5 cm/min. In certain embodiments, the stepwise change between the starting buffer and the elution buffer is applied to the polishing chromatography matrix at a volumetric flow rate corresponding to a superficial liquid velocity of about 2.0 cm/min. In certain embodiments, the stepwise change between the starting buffer and the elution buffer is applied to the polishing chromatography matrix at a volumetric flow rate corresponding to a superficial liquid velocity of about 2.5 cm/min. In certain embodiments, the stepwise change between the starting buffer and the elution buffer is applied to the polishing chromatography matrix at a volumetric flow rate corresponding to a superficial liquid velocity of about 3.0 cm/min. In certain embodiments, the stepwise change between the starting buffer and the elution buffer is applied to the polishing chromatography matrix at a volumetric flow rate corresponding to a superficial liquid velocity of about 3.5 cm/min. In certain embodiments, the stepwise change between the starting buffer and the elution buffer is applied to the polishing chromatography matrix at a volumetric flow rate corresponding to a superficial liquid velocity of about 4.0 cm/min. In certain embodiments, the stepwise change between the starting buffer and the elution buffer is applied to the polishing chromatography matrix at a volumetric flow rate corresponding to a superficial liquid velocity of about 5.0 cm/min. In certain embodiments, the stepwise change between the starting buffer and the elution buffer is applied to the polishing chromatography matrix at a volumetric flow rate corresponding to a superficial liquid velocity of about 6.0 cm/min. In certain embodiments, the stepwise change between the starting buffer and the elution buffer is applied to the polishing chromatography matrix at a volumetric flow rate corresponding to a superficial liquid velocity of about 7.0 cm/min. In certain embodiments, the stepwise change between the starting buffer and the elution buffer is applied to the polishing chromatography matrix at a volumetric flow rate corresponding to a superficial liquid velocity of about 8.0 cm/min. In certain embodiments, the stepwise change between the starting buffer and the elution buffer is applied to the polishing chromatography matrix at a volumetric flow rate corresponding to a superficial liquid velocity of about 9.0 cm/min. In certain embodiments, the stepwise change between the starting buffer and the elution buffer is applied to the polishing chromatography matrix at a volumetric flow rate corresponding to a superficial liquid velocity of about 10.0 cm/min.

In some embodiments, the mixture comprising the protein product and the contaminant is applied to the polishing chromatography matrix at a loading amount that is in excess of the maximum capacity of the polishing chromatography matrix, e.g., in an overload and elute chromatography mode. In some embodiments, the loading amount of the mixture that is applied to the polishing chromatography matrix is at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold higher than the maximum capacity of the polishing chromatography matrix. In certain embodiments, the loading amount of the mixture is at least about 1.5-fold higher than the maximum capacity of the polishing chromatography matrix. In certain embodiments, the loading amount of the mixture is at least about 2-fold higher than the maximum capacity of the polishing chromatography matrix. In certain embodiments, the loading amount of the mixture is at least about 3-fold higher than the maximum capacity of the polishing chromatography matrix. In certain embodiments, the loading amount of the mixture is at least about 4-fold higher than the maximum capacity of the polishing chromatography matrix. In certain embodiments, the loading amount of the mixture is at least about 5-fold higher than the maximum capacity of the polishing chromatography matrix. In certain embodiments, the loading amount of the mixture is at least about 6-fold higher than the maximum capacity of the polishing chromatography matrix. In certain embodiments, the loading amount of the mixture is at least about 7-fold higher than the maximum capacity of the polishing chromatography matrix. In certain embodiments, the loading amount of the mixture is at least about 8-fold higher than the maximum capacity of the polishing chromatography matrix. In certain embodiments, the loading amount of the mixture is at least about 9-fold higher than the maximum capacity of the polishing flow-through chromatography matrix.

In certain embodiments the loading amount is equal to the maximum capacity of the polishing chromatography matrix, e.g., in a bind and elute chromatography mode. In some embodiments, the loading amount is less than the maximum capacity of the polishing chromatography matrix, e.g., in a bind and elute chromatography mode.

I.C. Protein Products

The methods disclosed herein can be applied to any protein product. In some embodiments, the protein product is a therapeutic protein. In some embodiments, the therapeutic protein is selected from an antibody or an antigen-binding fragment thereof, an Fc fusion protein, an anticoagulant, a blood clotting factor, a bone morphogenetic protein, an engineered protein scaffold, an enzyme, a growth factor, a hormone, an interferon, an interleukin, and a thrombolytic. In particular embodiments, the protein product is an Fc fusion protein. In certain embodiments, the protein product is a recombinant protein.

In certain embodiments, the protein product is an antibody or an antigen binding fragment thereof. In some embodiments, the protein product is a chimeric polypeptide comprising an antigen binding fragment of an antibody. In certain embodiments, the protein product is a monoclonal antibody or an antigen binding fragment thereof (“mAb”). The antibody can be a human antibody, a humanized antibody, or a chimeric antibody. In certain embodiments, the protein product is a bispecific antibody.

In certain aspects of the present disclosure, the mixture comprising the protein product and the contaminant comprises a product of a prior purification step. In some embodiments, the mixture is the raw product of a prior purification step. In some embodiments, the mixture is a solution comprising the raw product of a prior purification step and a buffer, e.g., the starting buffer. In some embodiments, the mixture comprises the raw product of a prior purification step reconstituted in the starting buffer.

In some embodiments, the prior purification step comprises a chromatography. In some embodiments, the prior purification step comprises protein bind and elute chromatography. In some embodiments, the prior purification step comprises anion-exchange chromatography. In some embodiments, the prior purification step comprises a cation-exchange chromatography. In some embodiments, the prior purification step comprises an affinity chromatography. In some embodiments, the prior purification step comprises a mixed mode chromatography. In some embodiments, the prior purification step comprises a hydrophobic-interaction chromatography.

In particular embodiments, the prior purification step comprises a protein A chromatography. In some embodiments, the prior purification step comprises a protein A chromatography followed by low pH viral inactivation, pH neutralization, depth filtration or any combination thereof.

In certain embodiments, the mixture comprising the protein product and the contaminant comprises the elution product from a prior protein A chromatography, wherein the protein product comprises a monoclonal antibody or an antigen-binding fragment thereof.

In some embodiments, the source of the protein product is bulk protein. In some embodiments, the source of the protein product is a composition comprising protein product and non-protein components. The non-protein components can include DNA and other contaminants.

In some embodiments, the source of the protein product is from an animal. In some embodiments, the animal is a mammal such as a non-primate (e.g., cow, pig, horse, cat, dog, rat etc.) or a primate (e.g., monkey or human). In some embodiments, the source is tissue or cells from a human. In certain embodiments, such terms refer to a non-human animal (e.g., a non-human animal such as a pig, horse, cow, cat or dog). In some embodiments, such terms refer to a pet or farm animal. In specific embodiments, such terms refer to a human.

In some embodiments, the protein products purified by the methods described herein are fusion proteins. A “fusion” or “fusion protein” comprises a first amino acid sequence linked in frame to a second amino acid sequence with which it is not naturally linked in nature. The amino acid sequences which normally exist in separate proteins can be brought together in a fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some embodiments, the proteins purified by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In a specific embodiment, the antibody is a humanized monoclonal antibody. In another specific embodiment, the antibody is a human monoclonal antibody, preferably that is an immunoglobulin. In certain embodiments, an antibody described herein is an IgG1, or IgG4 antibody.

In some embodiments, the protein described herein is an “antigen-binding domain,” “antigen-binding region,” “antigen-binding fragment,” and similar terms, which refer to a portion of an antibody molecule which comprises the amino acid residues that confer on the antibody molecule its specificity for the antigen (e.g., the complementarity determining regions (CDR)). The antigen-binding region can be derived from any animal species, such as rodents (e.g., mouse, rat or hamster) and humans.

In some embodiments, the protein is an anti-LAG3 antibody, an anti-CTLA-4 antibody, an anti-TIM3 antibody, an anti-NKG2a antibody, an anti-ICOS antibody, an anti-CD137 antibody, an anti-KIR antibody, an anti-TGFβ antibody, an anti-IL-10 antibody, an anti-B7-H4 antibody, an anti-Fas ligand antibody, an anti-mesothelin antibody, an anti-CD27 antibody, an anti-GITR antibody, an anti-CXCR4 antibody, an anti-CD73 antibody, an anti-TIGIT antibody, an anti-OX40 antibody, an anti-PD-1 antibody, an anti-PD-L1 antibody, an anti-IL8 antibody, or any combination thereof. In some embodiments, the protein is Abatacept NGP. In other embodiments, the protein is Belatacept NGP.

In some embodiments, the protein is an anti-GITR (glucocorticoid-induced tumor necrosis factor receptor family-related gene) antibody. In some embodiments, the anti-GITR antibody has the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841, WO2016/057846, WO 2018/013818, or other anti-GITR antibody described or referred to herein, all of which are incorporated herein in their entireties.

In other embodiments, the protein is an anti-LAG3 antibody. Lymphocyte-activation gene 3, also known as LAG-3, is a protein which in humans is encoded by the LAG3 gene. LAG3, which was discovered in 1990 and is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right. Examples of anti-LAG3 antibodies include, but are not limited to, the antibodies in WO 2017/087901 A2, WO 2016/028672 A1, WO 2017/106129 A1, WO 2017/198741 A1, US 2017/0097333 A1, US 2017/0290914 A1, and US 2017/0267759 A1, all of which are incorporated herein in their entireties.

In some embodiments, the protein is an anti-CXCR4 antibody. CXCR4 is a 7 transmembrane protein, coupled to Gl. CXCR4 is widely expressed on cells of hemopoietic origin, and is a major co-receptor with CD4+ for human immunodeficiency virus 1 (HIV-1) See Feng, Y., Broeder, C. C., Kennedy, P. E., and Berger, E. A. (1996) Science 272, 872-877. Examples of anti-CXCR4 antibodies include, but are not limited to, the antibodies in WO 2009/140124 A1, US 2014/0286936 A1, WO 2010/125162 A1, WO 2012/047339 A2, WO 2013/013025 A2, WO 2015/069874 A1, WO 2008/142303 A2, WO 2011/121040 A1, WO 2011/154580 A1, WO 2013/071068 A2, and WO 2012/175576 A1, all of which are incorporated herein in their entireties.

In some embodiments, the protein is an anti-CD73 (ecto-5′-nucleotidase) antibody. In some embodiments, the anti-CD73 antibody inhibits the formation of adenosine. Degradation of AMP into adenosine results in the generation of an immunosuppressed and pro-angiogenic niche within the tumor microenvironment that promotes the onset and progression of cancer. Examples of anti-CD73 antibodies include, but are not limited to, the antibodies in WO 2017/100670 A1, WO 2018/013611 A1, WO 2017/152085 A1, and WO 2016/075176 A1, all of which are incorporated herein in their entireties.

In some embodiments, the protein is an anti-TIGIT (T cell Immunoreceptor with Ig and ITIM domains) antibody. TIGIT is a member of the PVR (poliovirus receptor) family of immunoglobin proteins. TIGIT is expressed on several classes of T cells including follicular B helper T cells (TFH). The protein has been shown to bind PVR with high affinity; this binding is thought to assist interactions between TFH and dendritic cells to regulate T cell dependent B cell responses. Examples of anti-TIGIT antibodies include, but are not limited to, the antibodies in WO 2016/028656 A1, WO 2017/030823 A2, WO 2017/053748 A2, WO 2018/033798 A1, WO 2017/059095 A1, and WO 2016/011264 A1, all of which are incorporated herein by their entireties.

In some embodiments, the protein is an anti-OX40 (i.e., CD134) antibody. OX40 is a cytokine of the tumor necrosis factor (TNF) ligand family. OX40 functions in T cell antigen-presenting cell (APC) interactions and mediates adhesion of activated T cells to endothelial cells. Examples of anti-OX40 antibodies include, but are not limited to, WO 2018/031490 A2, WO 2015/153513 A1, WO 2017/021912 A1, WO 2017/050729 A1, WO 2017/096182 A1, WO 2017/134292 A1, WO 2013/038191 A2, WO 2017/096281 A1, WO 2013/028231 A1, WO 2016/057667 A1, WO 2014/148895 A1, WO 2016/200836 A1, WO 2016/100929 A1, WO 2015/153514 A1, WO 2016/002820 A1, and WO 2016/200835 A1, all of which are incorporated herein by their entireties.

In some embodiments, the protein is an anti-IL8 antibody. IL-8 is a chemotactic factor that attracts neutrophils, basophils, and T-cells, but not monocytes. It is also involved in neutrophil activation. It is released from several cell types in response to an inflammatory stimulus.

In some embodiments, the protein is Abatacept (marketed as ORENCIA®). Abatacept (also abbreviated herein as Aba) is a drug used to treat autoimmune diseases like rheumatoid arthritis, by interfering with the immune activity of T cells. Abatacept is a fusion protein composed of the Fc region of the immunoglobulin IgG1 fused to the extracellular domain of CTLA-4. In order for a T cell to be activated and produce an immune response, an antigen presenting cell must present two signals to the T cell. One of those signals is the major histocompatibility complex (MHC), combined with the antigen, and the other signal is the CD80 or CD86 molecule (also known as B7-1 and B7-2).

In some embodiments, the protein is Belatacept (trade name NULOJIX®). Belatacept is a fusion protein composed of the Fc fragment of a human IgG1 immunoglobulin linked to the extracellular domain of CTLA-4, which is a molecule crucial in the regulation of T cell costimulation, selectively blocking the process of T-cell activation. It is intended to provide extended graft and transplant survival while limiting the toxicity generated by standard immune suppressing regimens, such as calcineurin inhibitors. It differs from abatacept (ORENCIA®) by only 2 amino acids.

I.D. Column Switching

In certain embodiments, a column switching method is employed after the mixture comprising the protein product and the contaminant is contacted with the polishing chromatography matrix, wherein an auxiliary column containing a second polishing chromatography matrix is inserted into the mobile phase flow path downstream from the polishing chromatography matrix.

In some embodiments, the column switching mode is employed before the mixture comprising the protein product and the contaminant is contacted with the polishing chromatography matrix, wherein the mixture is applied to a main column containing a bind-and-elute chromatography matrix, and wherein the polishing chromatography matrix is inserted into the mobile phase flow path downstream from the bind-and-elute chromatography matrix.

In another embodiment, column switching is employed after the loading step so that an small auxiliary column containing fresh column packing (with the column packing being either the same type or a different type compared to the main column) is inserted into the flow path after the main column so the elution from the polishing chromatography matrix can be performed more efficiently. This alternative version of the invention employs overloading during the loading step so that product exits the main column and is collected during this step. Loading is then followed by a “pseudo” bind and elute step made possible by inserting the auxiliary column into the flow path during the chromatofocusing elution step.

### EXAMPLES

### Example 1

Chromatofocusing is a high-resolution protein separation method that combines features of ion-exchange chromatography and isoelectric focusing. Described herein are methods of separating a protein product from one or more contaminants that retain the concept employed in chromatofocusing of using a retained gradient inside a chromatographic column to achieve a separation. However, the described methods are applicable to industrial-scale purification of a single target protein by using simple mixtures of a relatively small number of buffering species (instead of a polyampholyte buffer) to produce a pH gradient composed of a small number of individual, stepwise pH transitions (instead of a gradual pH gradient). In this way the objective of efficiently purifying a single target protein at a large scale can be accomplished.

Anion-exchange column is initially equilibrated with a starting buffer, a feed sample is subsequently introduced onto the column, and then a single stepwise change is implemented from the starting buffer to the elution buffer. Due to the interactions of the various species present in the starting and elution buffers, the stepwise change in composition introduced into the column produces a self-generated gradient inside the column that may consist of both pH and ionic strength changes as well as multiple retained and unretained composition fronts that propagate through the column as discrete entities that are separated by composition plateaus. The retained fronts produced by the method are self-sharpening (or stepwise) in nature. Proteins present in the feed sample then locate themselves at characteristic positions on the gradient and become separated as they travel to the column exit.

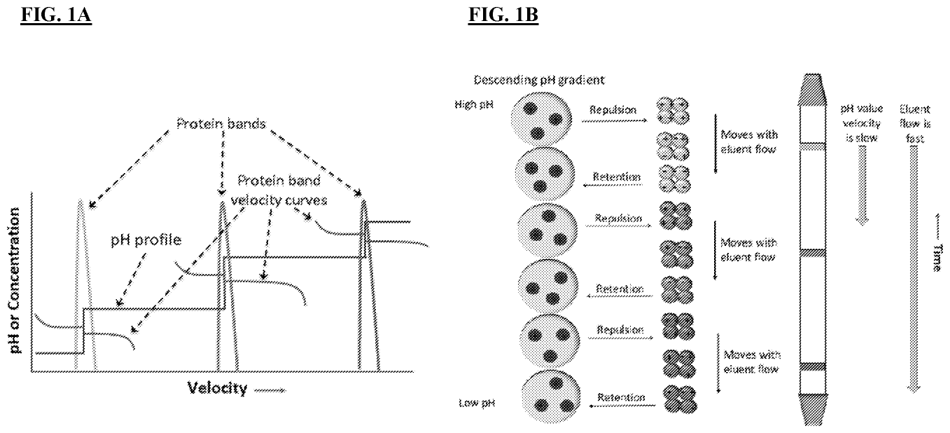

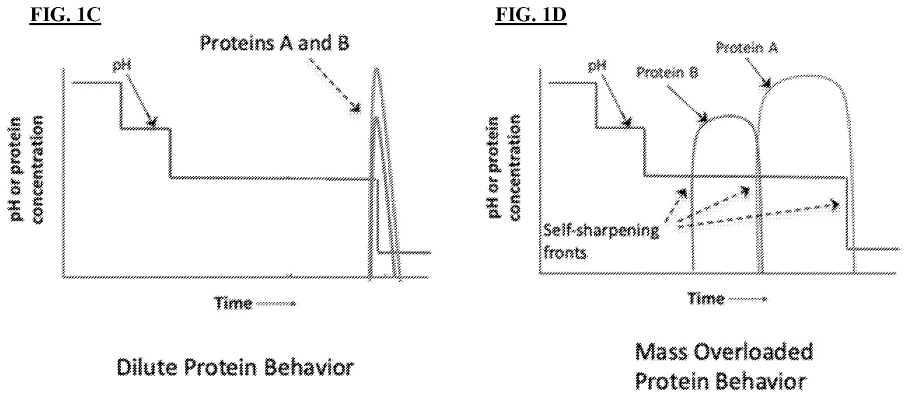

Chromatofocusing can be used as a polishing step following protein purification, using a low or high loading volume. When the retained-gradient polishing chromatography method is employed at low protein loading amounts (FIG. 1A), each protein present elutes from the column at its respective apparent isoelectric point (pIₐₚₚ). A focusing effect is produced such that proteins focus into narrow bands at their apparent isoelectric point in the column effluent (FIG. 1B). When using a pH gradient consisting of multiple stepwise pH fronts (as opposed to a continuous, gradual pH gradient) it will sometimes be the case that two closely related proteins will co-elute at the same pH front (FIG. 1C). For mass overloaded conditions where interactions of proteins in the adsorbed state is a factor, the focusing pH front functions as a protein displacer and resolves proteins even with very similar pI values (and therefore very similar pIₐₚₚ values) (FIG. 1D).

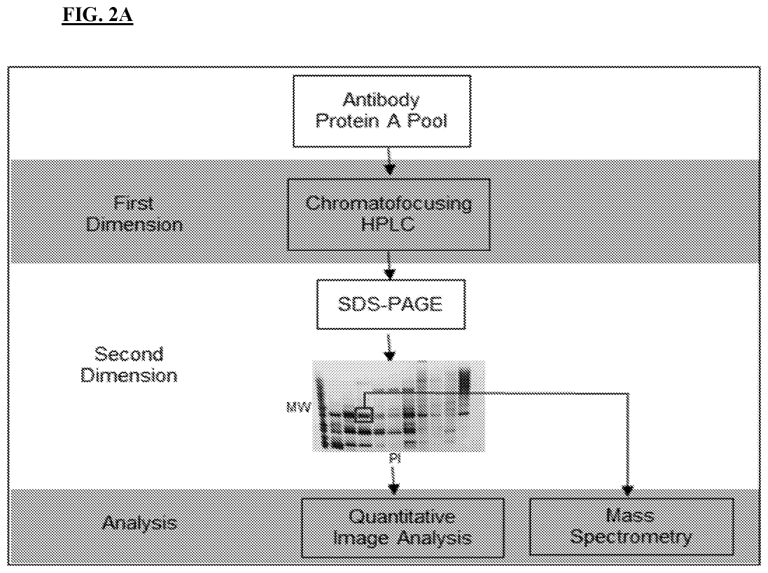

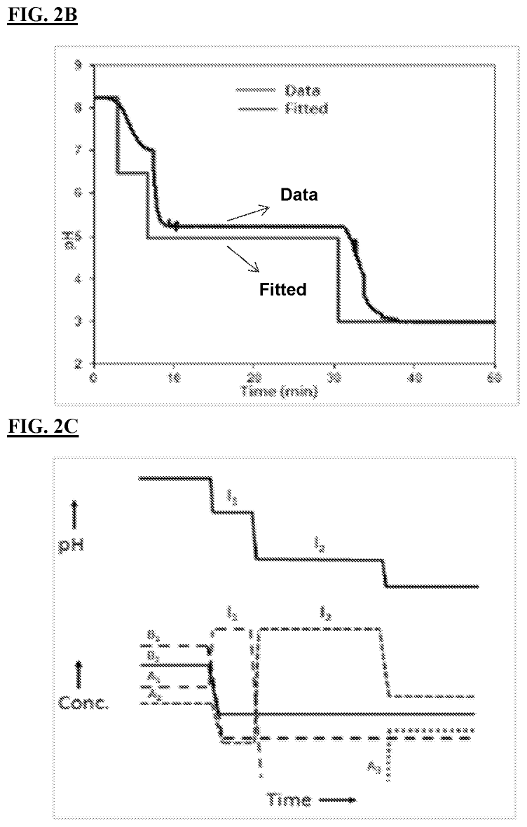

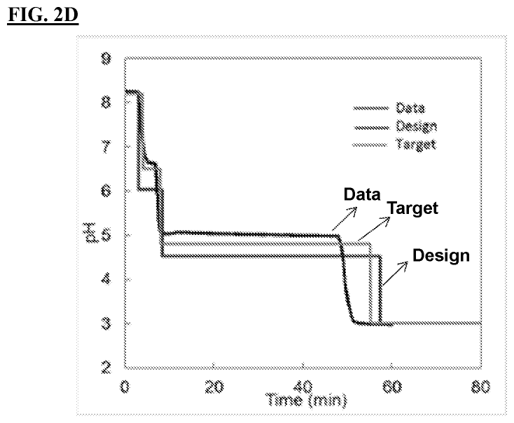

A 2D analytical fractionation tool for polishing process development was used to determine the quantity of both the impurities and monoclonal antibody product in a protein A pool as well as the “apparent” isoelectric points of these species that pertain to the method (FIG. 2A, top row). A specific anion-exchange column packing and a generic pH gradient consisting of ten small self-sharpening pH fronts, i.e., a ten-step anion-exchange chromatofocusing pH gradient, was used as the first dimension of the 2D method (FIG. 2A, second row). Column effluent fractions from this first dimension were then collected from each of the ten pH fronts, and each of these fractions was subjected to high sensitivity silver-stained SDS-PAGE to form a 2D map of the components of the protein A pool (FIG. 2A, third row). This method was used to select pH gradient shapes and column packings for process-scale chromatofocusing. The method permits identifying specific impurity proteins using the size and apparent pI obtained by the 2D gel, or by using MS information obtained from excised gel bands. From the overlap of product and HCPs on the gel, predictions can be made about the behavior of chromatofocusing as a polishing method (FIG. 2A, bottom row). Computer-aided parameter fitting and buffer design for chromatofocusing based on local-equilibrium theory were employed as an additional tool for rational process development (FIGS. 2B-2D).

By comparing the relative positions of the gel bands for the host cell proteins (HCPs) and antibody product components, a pH gradient for a polishing process can be selected that applies to the specific column packing, protein A pool, and purification objective under consideration.

The starting and elution buffers corresponding to the selected pH gradient are then designed using software based on a local-equilibrium principle. Briefly,

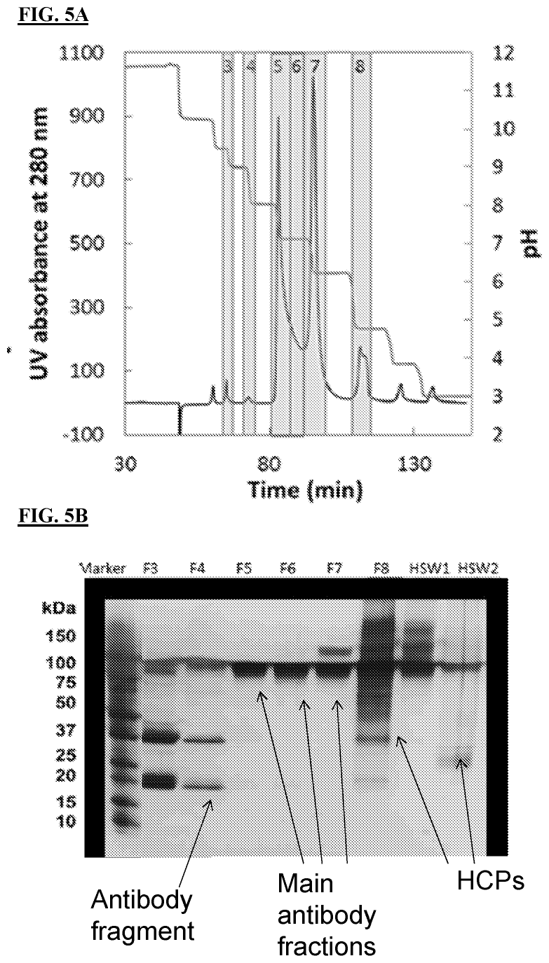

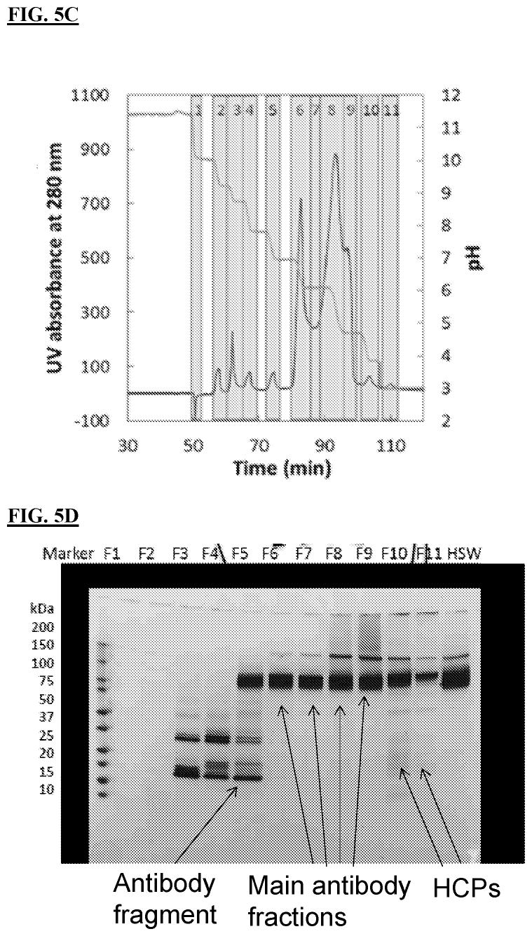

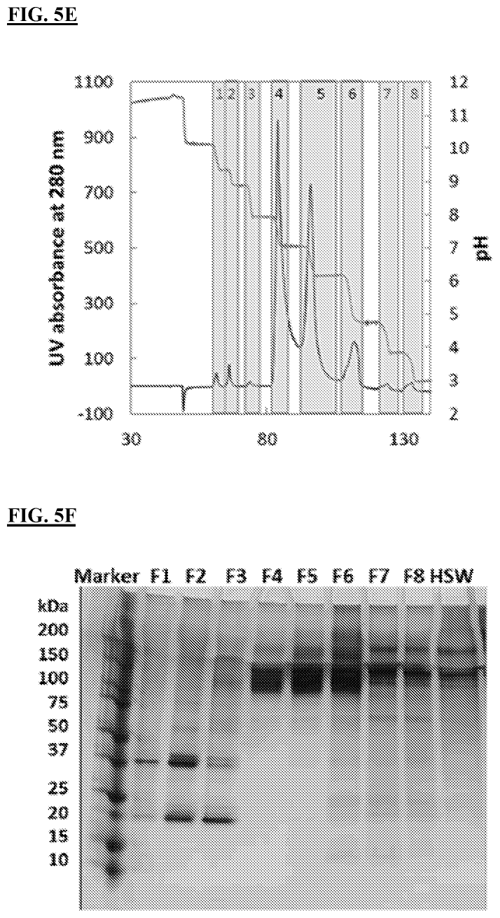

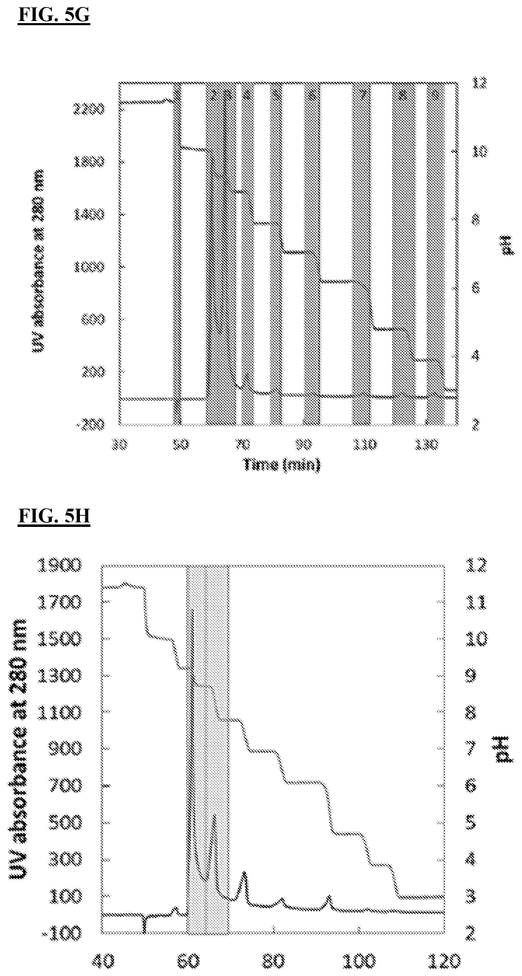

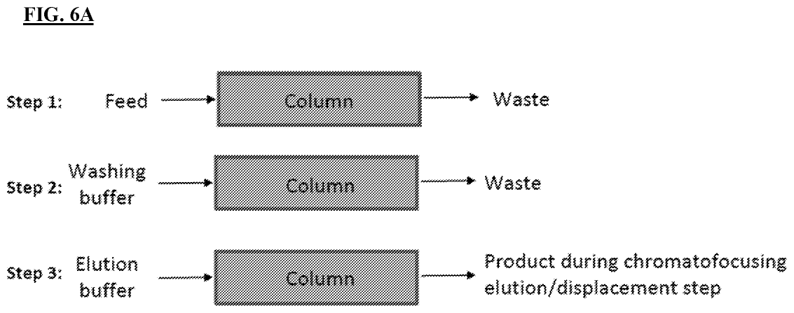

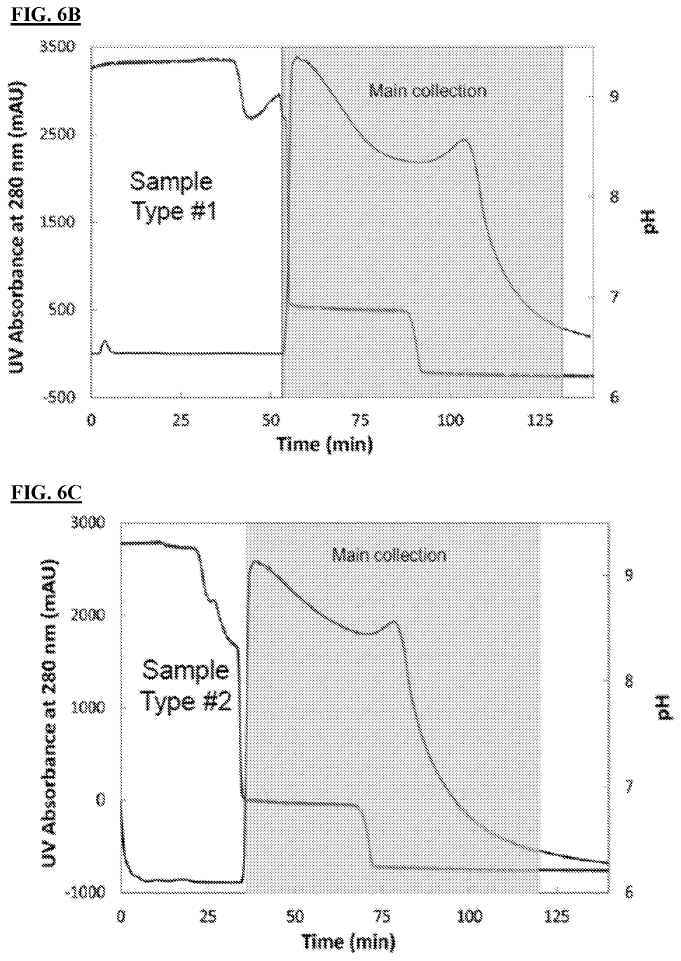

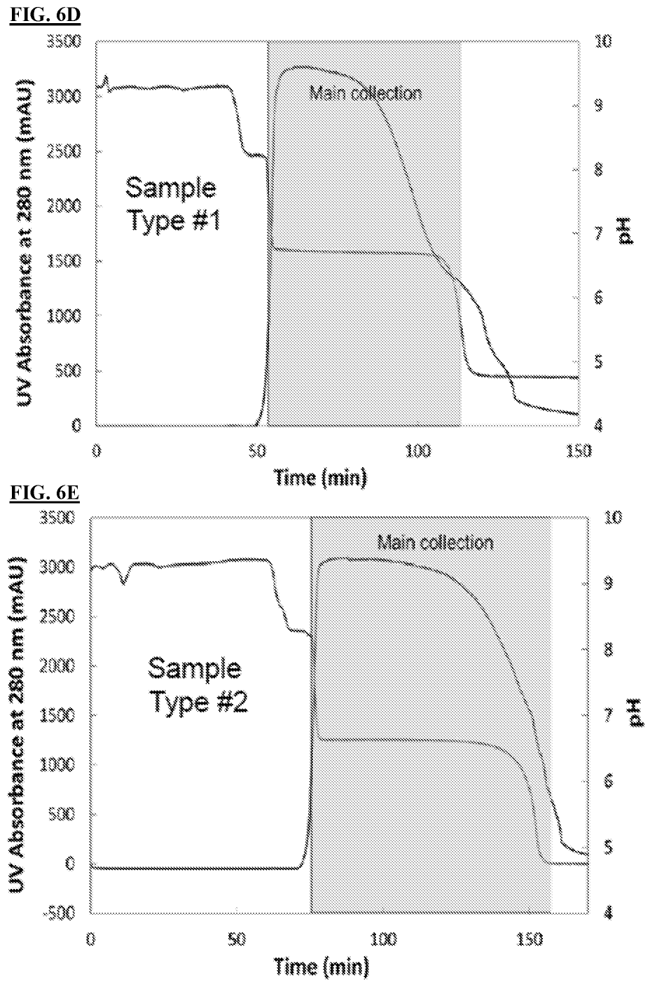

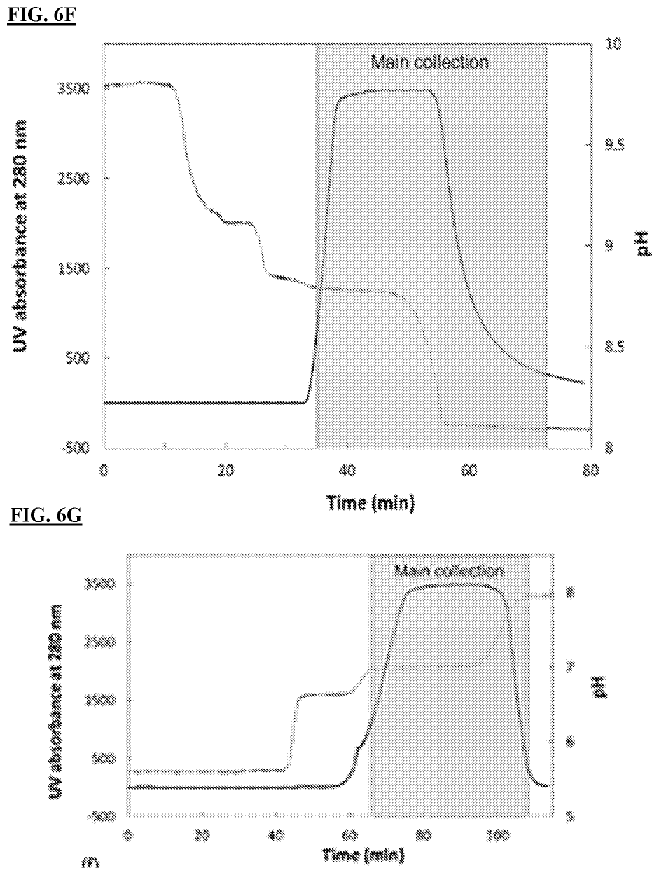

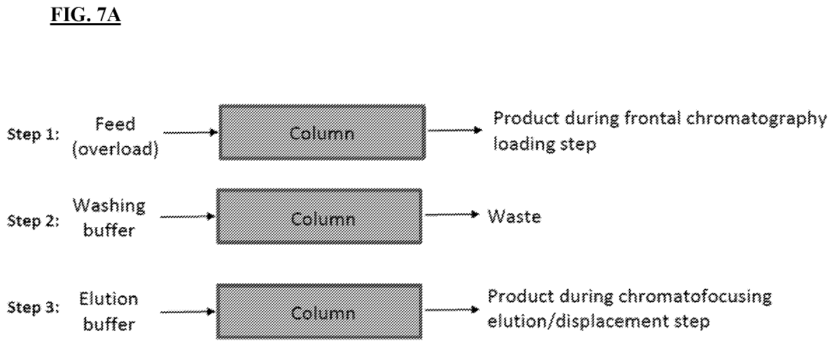

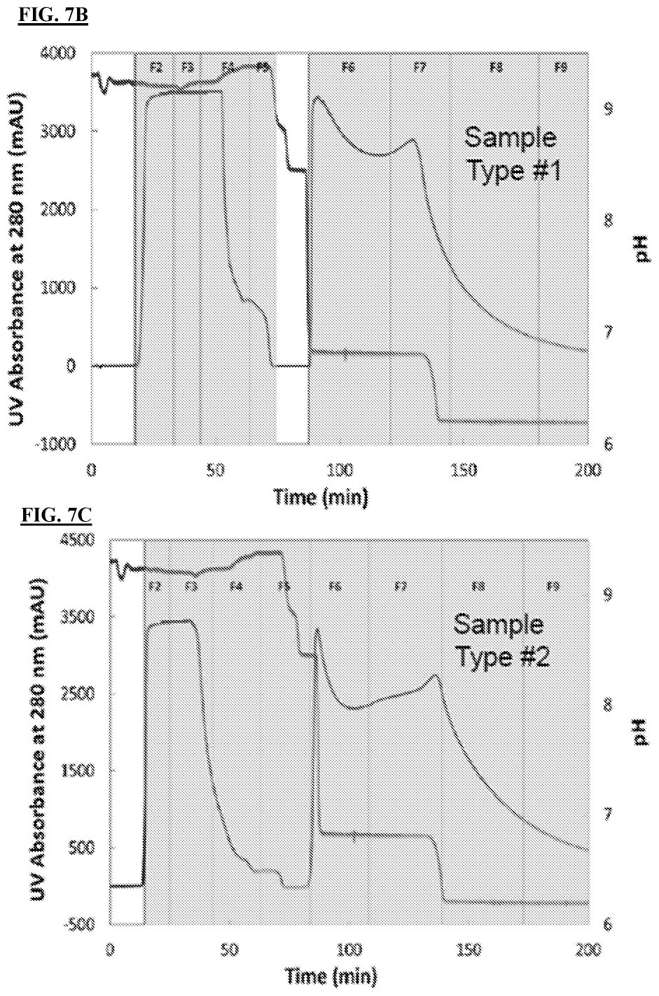

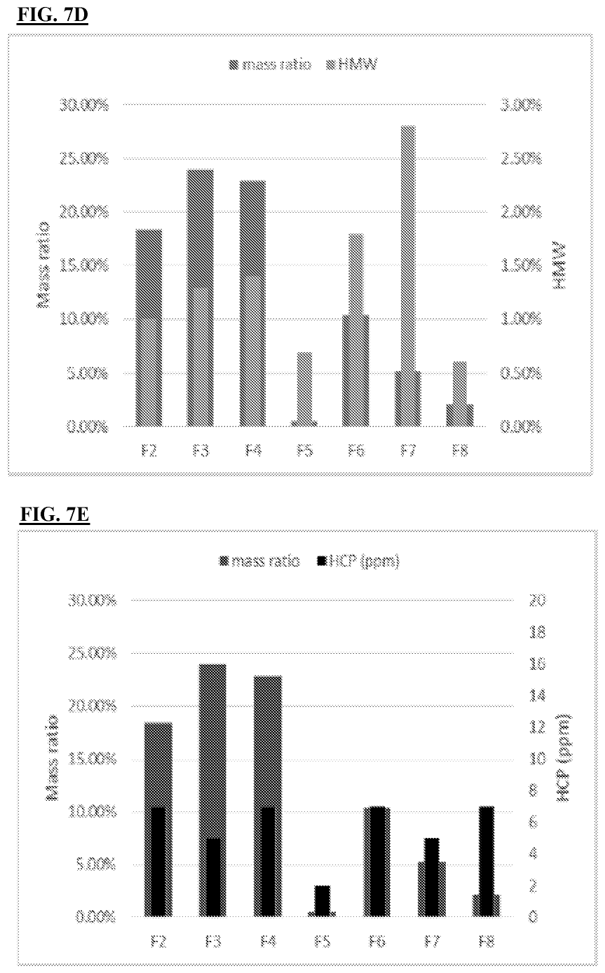

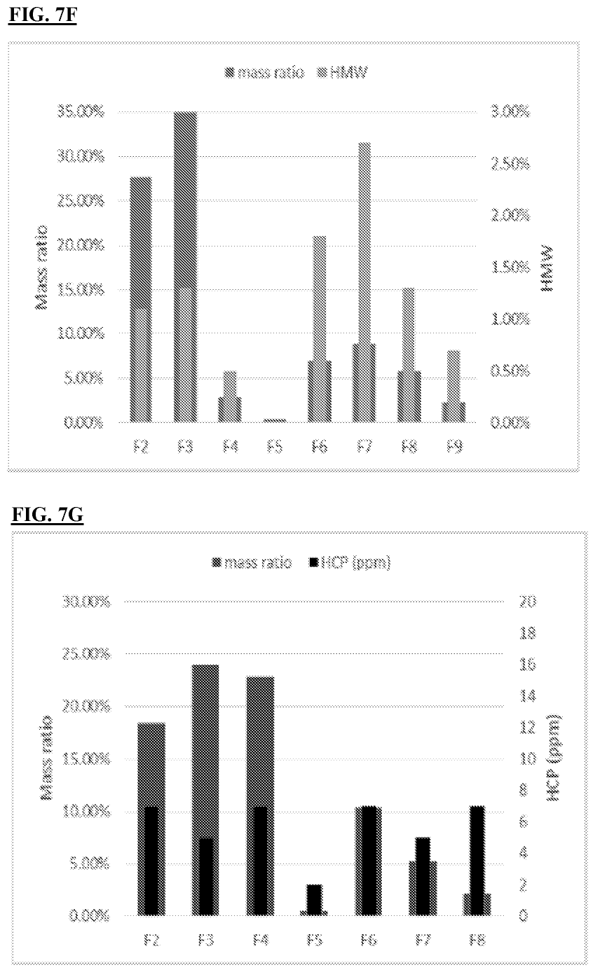

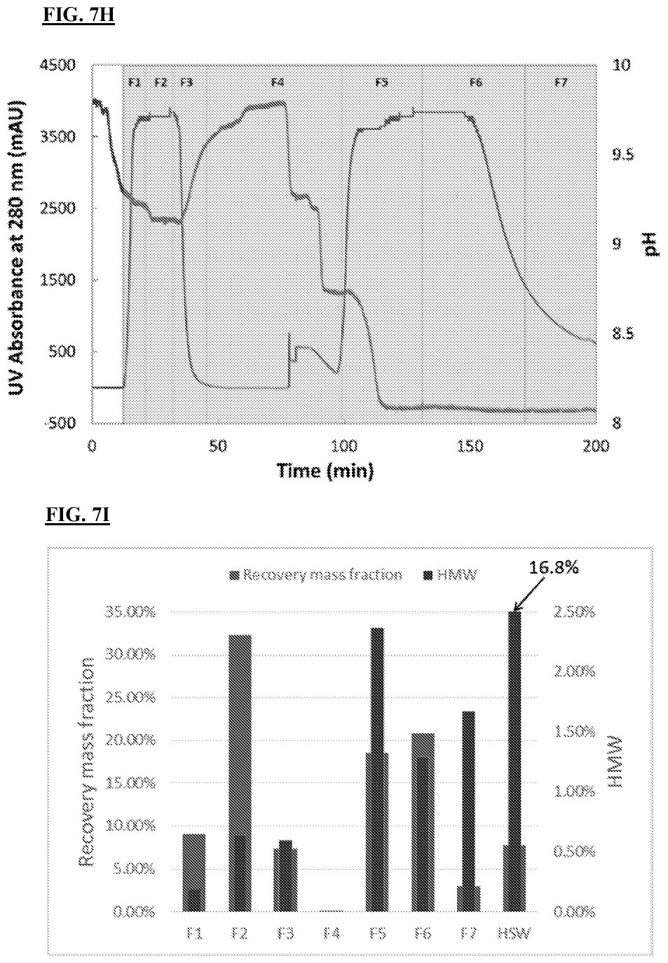

FIG. 6B shows the chromatogram and pH gradient designed as described above. Because of the limitation of the column packing dynamic binding capacity (DBC), when a high mass loading is desired during the feed loading step, the anion-exchange chromatofocusing method can alternatively be operated in the overload and elute mode (FIG. 7C). According to the 2D gel results in FIGS. 5A-5B, most of the impurities in the protein A pool considered here are more acidic and bind stronger to the column packing employed than the monoclonal antibody product. Consequently, in the overload and elute mode of operation the column effluent from the loading step after the feed material breaks through will be highly purified. When operating the chromatofocusing polishing step in the overload and chromatofocusing elution mode, the fractions pooled to produce the purified product will consist of the breakthrough product fraction obtained during the loading step where the loading amount is in excess of the column packing DBC together with appropriately chosen fractions of the monoclonal antibody product eluted during the chromatofocusing elution step.

One characteristic of the overload and chromatofocusing elution process is that the loading conditions employed are fully in the nonlinear equilibrium regime in order to exploit nonlinear interactions between adsorbed species. These conditions will generally correspond to a value for the partition coefficient that is greater than 5 but less than the maximum value possible for the partition coefficient, which is often near 100. Another characteristic is that the feed loading step is stopped before impurities such as HCPs and aggregates start to breakthrough into the column effluent. Furthermore, there typically is enough adsorbed monoclonal antibody product after the feed loading step to justify recovering purified fractions during the subsequent chromatofocusing elution step. Compared with traditional overload and elute chromatography which uses an unretained pH or salt gradient to elute the bound monoclonal antibody product, the elution step in the chromatofocusing method employs a retained pH gradient to achieve self-sharpening (and therefore also retained) concentration fronts to remove bound monoclonal antibody product in a more efficient manner. An experimental chromatogram and pH gradient for the example mAb protein A pool purification in overload and elute mode is shown in FIG. 7C.

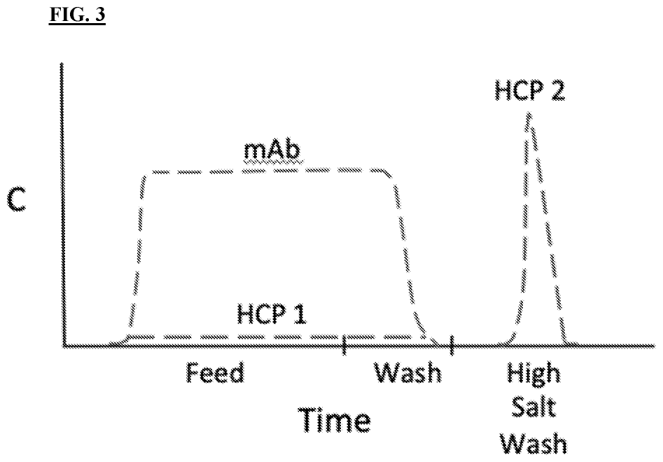

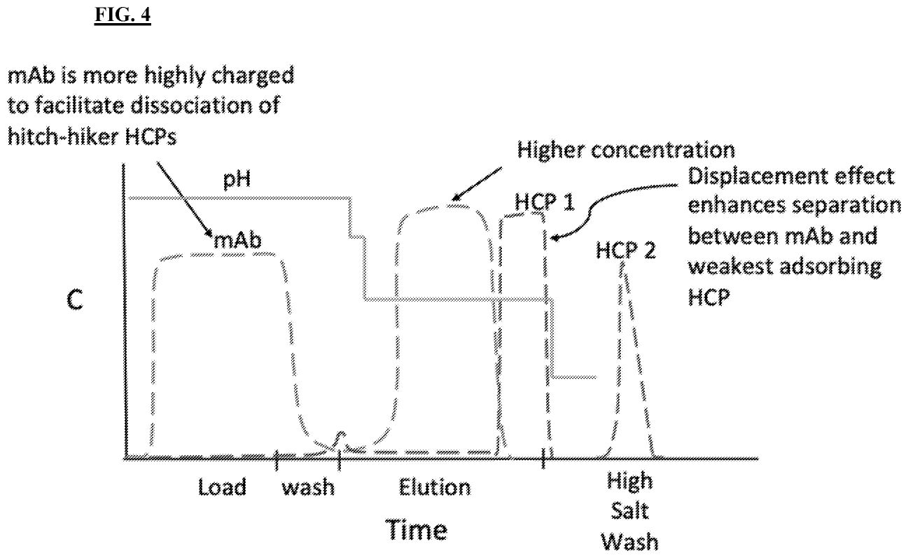

As a comparison, the effluent protein concentration profiles for traditional weak partitioning chromatography are shown qualitatively in FIG. 3. For this method, the loading conditions are in the linear adsorption equilibrium regime. Since it is assumed in this method that the impurities that are present are more strongly bound to the column packing than the monoclonal antibody product, just a small amount of HCPs co-elute with the monoclonal antibody product during the loading and washing step, and the majority of the HCPs remain bound to the column during these steps along with relatively minor amounts of the monoclonal antibody product. A subsequent high salt strip is then used to remove the bound impurities and monoclonal antibody from the column to form a waste product. It is possible to obtain a sharp product desorption front during the washing step as shown in FIG. 3 due to the linear adsorption equilibrium regime that is employed. In contrast, if nonlinear adsorption equilibrium is employed during the loading and wash steps in this process, then a long product tail contaminated with impurities will occur during the wash step, or a large amount of monoclonal antibody product will remain bound to column after the wash step, or a combination of these undesirable behaviors will occur.

In the traditional weak partitioning chromatography, only the physical adsorptive interactions between the column packing and species being separated are employed, as opposed to interactions directly between the species being separated. Therefore, for certain combinations of isoelectric points and quantities present of the product monoclonal antibody and the HCP and aggregate impurities as well as purification objectives, it will be more effective to operate the loading step and the subsequent steps of the polishing process in the nonlinear adsorption equilibrium regime since more material will bind onto the column packing in this case and there will consequently be more interactions between adsorbed bound species that can potentially assist the separation being performed. In particular, for the case of anion-exchange polishing, operating in the nonlinear loading regime will usually mean a higher loading pH is used so that the monoclonal antibody product is more highly negatively charged. This higher negative charge is likely to facilitate the dissociation of so-called hitch-hiker HCPs from the product monoclonal antibody since these HCP species are typically acidic in nature and therefore also negatively charged. This dissociation is likely to lead to a more effective removal of these types of HCPs from the monoclonal antibody product. However, when operating in the nonlinear adsorption equilibrium regime the conditions have to be properly selected to avoid long product tailing during the washing step and subsequent.

The protein concentration profiles for the chromatofocusing method using the overload and elute mode are shown qualitatively in FIG. 4. As shown, when the process is properly designed, all the composition transitions in the system have minimal tailing behavior. This is because of the following four possible reasons: (1) the transition is inherently sharp in nature due to the shape of the nonlinear adsorption isotherms that apply (which is the case for fronts 1 and 3 in FIG. 4 where thermodynamically self-sharpening fronts are formed); (2) the transition involves a desorption step where the adsorption equilibrium isotherm is highly nonlinear and therefore nearly irreversible (which is the case for front 2 in FIG. 4); (3) the transition involves a simultaneous pH change so that it is part of the chromatofocusing pH gradient (which is the case for front 5 in FIG. 4); and (4) the transition is part of a displacement train of rectangular species bands (which is the case for front 4 in FIG. 4). Consequently very small amounts of HCPs will co-elute with the monoclonal antibody product during all the steps used in the process shown in FIG. 4 if proper fractions are collected. This process permits a different pH to apply for the loading step and the chromatofocusing elution step since different performance considerations apply to these two steps.

Bind and Elute Chromatography Mode

To accomplish the bind and elute chromatography mode, after equilibrating the column with the loading buffer, the feed sample is loaded close to the product dynamic binding capacity (DBC) of the column packing, and then a wash step is performed with the equilibration buffer. Then a single stepwise change is implemented from the starting buffer to the elution buffer. A retained pH gradient is then formed inside the column and most of the antibody product is eluted within one or two pH fronts while most impurities are eluted using other pH fronts or remain adsorbed on the column. In this way the monoclonal antibody product is separated from the impurities in the protein A pool.

Overload and Elute Chromatography Mode

Because of the limitation of the loading capacity of the chromatography column packing for the monoclonal antibody product, the purification of the monoclonal antibody protein A pool using the chromatofocusing method is operated in the overload and elute chromatography mode. After equilibrating the column with the loading buffer, the feed material is loaded up to, and beyond, the DBC of the column packing until impurities start to break through in the column effluent. Then a wash step is performed with the loading buffer until the UV absorbance returns to near baseline level. Although the product starts to break through in the column effluent at some point after continually loading the feed material as just described, most impurities continue to bind during the breakthrough phase. Consequently the product pool collected during the load phase is highly pure. The remaining product bound onto the column can be recovered in selected effluent fractions by eluting with the chromatofocusing pH gradient while at the same time most of the impurities will either elute in different effluent fractions or will remain bound to the column packing so that they can be washed out of the column with a high salt buffer after the chromatofocusing step.

Buffers

The buffer composition of the equilibration buffer and the elution buffer are designed so that by simply performing a step-wise change from an equilibration buffer to an elution buffer, a self-generated pH gradient is produced inside the column. The protein A pool was either diafiltered with the equilibration buffer or adjusted to the appropriate pH and conductivity before loading onto the chromatofocusing column. A washing step with the equilibration buffer was performed after loading to wash out the product remained in the interparticle space of the column packing as well as to presaturate the column packing with the equilibration buffer components. In addition to anion-exchange chromatofocusing as described in this document, the use of cation-exchange chromatofocusing is also a useful option depending on the conditions that apply.

### Example 2

Antibody Preparation

An antibody feed sample obtained from a protein A chromatography product pool was diafiltered with starting buffer (9.4 mM AMPSO, 20.5 mM MOPS, titrate with NaOH to pH=9.2) using an Amicon 50 kDa Ultra-15 centrifugal filter. The sample was then diluted to approximately 10 mg/mL antibody with the starting buffer and then filtered with a nylon syringe filter having 0.2 μm pores.

Bind and Elute Mode

A 0.7 mL Omnifit glass column containing the TOYOPEARL SuperQ-650S column packing material was equilibrated with the starting buffer at a flow rate of 0.35 mL/min for 40 minutes. The column was then loaded with approximately 3 mL of the diafiltered antibody, where the antibody was obtained from a protein A chromatography product pool, which yielded a load of approximately 42 mg antibody/mL of packed column.

After the feed sample was loaded, the column was washed with the starting buffer for 10 minutes. Both the loading and washing flow rate were conducted at 0.35 mL/min.

A retained chromatofocusing pH gradient was created by performing a stepwise change between the starting buffer and the elution buffer (12 mM MOPS, 10 mM MES, titrate with NaOH to pH=6.2) at the entrance of the column with the flow rate set at 0.2 mL/min. The UV absorbance of the column effluent was monitored at 280 nm. The antibody peak was identified by its UV absorbance reading and was collected in several fractions.

After the chromatofocusing elution step, a high salt wash step was performed using 1.5 M NaCl in order to elute strongly bound impurities and residual antibody products from the column. As described above, the flow rates of the buffers used in the loading and washing steps were typically higher in comparison to the flow rates of the buffers used for the chromatofocusing elution step in order to reduce the overall time for the procedure while also maintaining good performance for the chromatofocusing elution step.

Overload and Elute Mode

A 0.7 mL Omnifit glass column containing the SuperQ-650S column packing material was equilibrated with the starting buffer at a flow rate of 0.35 mL/min for 40 minutes. The column was then loaded with approximately 10 mL of diafiltered antibody, where the antibody was obtained from a protein A chromatography product pool, which yielded a load of approximately 142 mg antibody/mL of packed column. During the loading step the UV absorbance of the column effluent was monitored at 280 nm, and column effluent fractions were collected when the antibody began to exit the column as determined by the UV absorbance reading. After the feed sample was loaded, the column was washed with the starting buffer for 30 minutes to wash out any antibody product that remained in the interparticle space of the column packing material. The collection of column effluent fractions was discontinued when the UV absorbance level returned to near baseline level during the washing step. The flow rate of the buffer was 0.35 mL/min during both the loading and washing steps.

After the loading and washing steps, the remaining antibody product that was bound onto the column packing material was eluted using a retained chromatofocusing pH gradient that was produced by performing a stepwise change between the starting and elution buffers. A flow rate of 0.2 mL/min was employed during the chromatofocusing elution step, and the UV absorbance of the column effluent was monitored at 280 nm in order to identify column effluent fractions containing antibody. The entire antibody product pool consisted of both the antibody fractions collected during the loading and washing steps and the antibody fractions collected during the chromatofocusing elution step.

After the chromatofocusing elution step, a high salt wash was performed using 1.5 M NaCl to elute strongly bound impurities and residual antibody products from the column.

Auxiliary Column and Column Switching Method

An auxiliary column (either 0.7 or 0.35 mL in volume) contained the same SuperQ-650S, GigaCap, or FRACTOGEL® SO₃″ column packing material as the main column (which was 0.7 mL in volume). Both the main column and the auxiliary columns were first equilibrated with the starting buffer at a flow rate of 0.35 mL/min for 40 minutes. Then, 10 mL of diafiltered antibody sample, where the antibody was obtained from a protein A chromatography product pool, was loaded onto the main column to yield a load of approximately between about 100 mg antibody/mL to 142 mg antibody/mL of packed column. After the feed sample was loaded, the main column was washed with the starting buffer for 30 minutes to wash out the antibody product that remained in the interparticle space of the column packing. During the loading and washing steps, the UV absorbance of the column effluent was monitored at 280 nm. The collection of antibody product fractions began during the loading step and when the antibody began to exit the column as determined by the UV absorbance reading. The collection of antibody production fractions was discontinued when the UV absorbance reading returned to near baseline level during the washing step. The flow rate of the buffers during both the loading and washing steps was 0.35 mL/minute.

After the washing step, the auxiliary column with fresh column packing was inserted into the flow path after (i.e., downstream from) the main column. The remaining antibody product bound onto the main column was then eluted by using a retained chromatofocusing pH gradient produced by performing a stepwise change between the starting and elution buffers. The pH gradient traveled first through the main column and then through the auxiliary column. The entire antibody product pool consisted of both the antibody fractions collected during the loading and washing steps and the antibody fractions collected during the chromatofocusing elution step.

After the chromatofocusing elution step, a high salt wash was performed using 1.5 M NaCl to elute strongly bound impurities and residual antibody products from the column.

Auxiliary Column Without Column Switching Method

An auxiliary column (either 0.7 or 0.35 mL in volume) contained the same SuperQ-650S, GigaCap, or FRACTOGEL® SO₃″ column packing material as the main column (which was 0.7 mL in volume). The main column and the auxiliary columns were first connected together and equilibrated with the starting buffer at a flow rate of 0.35 mL/min for 40 minutes. Then, 10 mL of diafiltered antibody sample, where the antibody was obtained from a protein A chromatography product pool, was loaded onto the connected main column and auxiliary column. A total of approximately 100 mg to 130 mg antibody was loaded onto the two columns. After the feed sample was loaded onto both columns, the two columns were washed with the starting buffer for 30 minutes to wash out the antibody product that remained in the interparticle space of the column packing. During the loading and washing steps, the UV absorbance of the column effluent was monitored at 280 nm. The collection of antibody product fractions began during the loading step and when the antibody began to exit the column as determined by the UV absorbance reading. The collection of antibody production fractions was discontinued when the UV absorbance reading returned to near baseline level during the washing step. The flow rate of the buffers during both the loading and washing steps was 0.35 mL/minute.

After the loading and washing steps, the remaining antibody product that was bound onto the two columns was eluted using a retained chromatofocusing pH gradient that was produced by performing a stepwise change between the starting and elution buffers. The pH gradient traveled first through the main column and then through the auxiliary column. The entire antibody product pool consisted of both the antibody fractions collected during the loading and washing steps and the antibody fractions collected during the chromatofocusing elution step.

### Example 3

Strong anion-exchange chromatofocusing with different column packings was employed as the first separation dimension of two different antibody protein A products (i.e., either sample type #2 or sample type #1 from Antibody B and Antibody D protein A pools after low pH virus inactivation, pH neutralization, and depth filtration (positively charged)). In particular, Antibody B, sample type #1 was subjected to strong anion-exchange chromatofocusing with Super Q column packing (FIG. 5A, UV aborbance; FIG. 5B SDS-PAGE results). As shown in FIGS. 5A-5B, fractions F5-F7 contained mainly the monoclonal antibody product while the impurities in the protein A pool were largely present in other fractions (in particular fraction F8). Antibody B, sample type #1 was subjected to strong anion-exchange chromatofocusing with GigaCap column packing (FIG. 5C, UV aborbance; FIG. 5D SDS-PAGE results). Antibody B, sample type #2 was subjected to strong anion-exchange chromatofocusing with Super Q column packing (FIG. 5E, UV aborbance; FIG. 5F SDS-PAGE results). Antibody D, sample type #1 was subjected to strong anion-exchange chromatofocusing with Super Q column packing (FIG. 5G) and GigaCap column packing (FIG. 5H). These results indicate that the sensitivity for detecting HCPs using the 2D method is greatly improved as compared with directly loading the protein A product onto a single gel lane provided that the HCPs and antibody do not co-elute in the chromatofocusing step of the 2D method. Most HCPs contained in the samples were likely “hitchhiker” HCPs. These results indicate that anion-exchange chromatofocusing has the potential to disrupt the mAb-HCP interaction. These results further show that the antibody elutes at different pH fronts for different column packings, suggesting that different pH gradients could be designed for the chromatofocusing polishing step for different column packings.

### Example 4

The results of anion-exchange chromatofocusing were applied to the polishing of antibodies from a protein A pools using the bind and elute mode (FIG. 6A). FIGS. 6B and 6C show results from anion-exchange chromatofocusing of Antibody B from a protein A pool using the bind and elute mode with strong anion exchanger Super Q column packing. The parameters and results are summarized in Table 1.

FIGS. 6D and 6E show results from anion-exchange chromatofocusing applied to the polishing of Antibody B from a protein A pool using the bind and elute mode with strong anion exchanger GigaCap column packing. The parameters and results are summarized in Table 2.

FIG. 6F shows results from anion-exchange chromatofocusing applied to the polishing of Antibody D from a protein A pool using the bind and elute mode with strong anion exchanger GigaCap column packing. The parameters and results are summarized in Table 3.

FIG. 6G shows results from cation-exchange chromatofocusing applied to the polishing of Antibody B from a protein A pool using the bind and elute mode with strong cation exchanger FRACTOGEL® SO₃⁻ column packing.

### Example 5

The results of anion-exchange chromatofocusing were applied to the polishing of Antibody B from protein A pools using the overload and elute mode with strong anion-exchange Super Q column packing using a single column (FIGS. 4 and 7A). Relative amounts of HCPs and HMWs for each case are shown in FIGS. 7D-7G. The properties of the column and feed sample used are summarized in Table 4.

TABLE 4

| | | | | Feed | | |
|---|---|---|---|---|---|---|
| | | | | material | | |
| | | | Mass | HCP | Feed | |
| Sample | Number of | Column | loaded | amount | material | |
| type | columns | size | (mg) | (ppm) | HMW | FIG. |
| #1 | one | 0.7 mL | 136 | 47 | 2.5% | FIGS. 7B, |
| | | | | | | 7D, 7E |
| #2 | one | 0.7 mL | 100 | 1634 | 3.3% | FIGS. 7C, |
| | | | | | | 7F, 7G |

The results of anion-exchange chromatofocusing applied to the polishing of Antibody D from a protein A pool using the overload and elute mode with strong anion-exchange GigaCap column packing using a single column are shown in FIGS. 7H-7I. The relative amounts of HMWs are shown in FIG. 7I. The properties of the column and feed sample used are summarized in Table 5.

TABLE 5

| | | | | Feed | | |
|---|---|---|---|---|---|---|
| | | | | material | | |
| | | | Mass | HCP | Feed | |
| Sample | Number of | Column | loaded | amount | material | |
| type | columns | size | (mg) | (ppm) | HMW | FIG. |
| #1 | one | 0.7 mL | 126 | 6 | 2.3% | FIGS. 7H-7I |

### Example 6

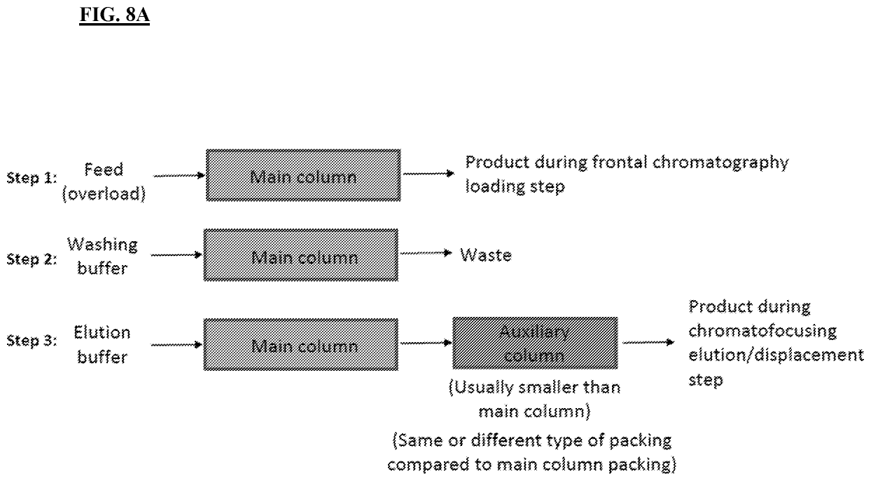

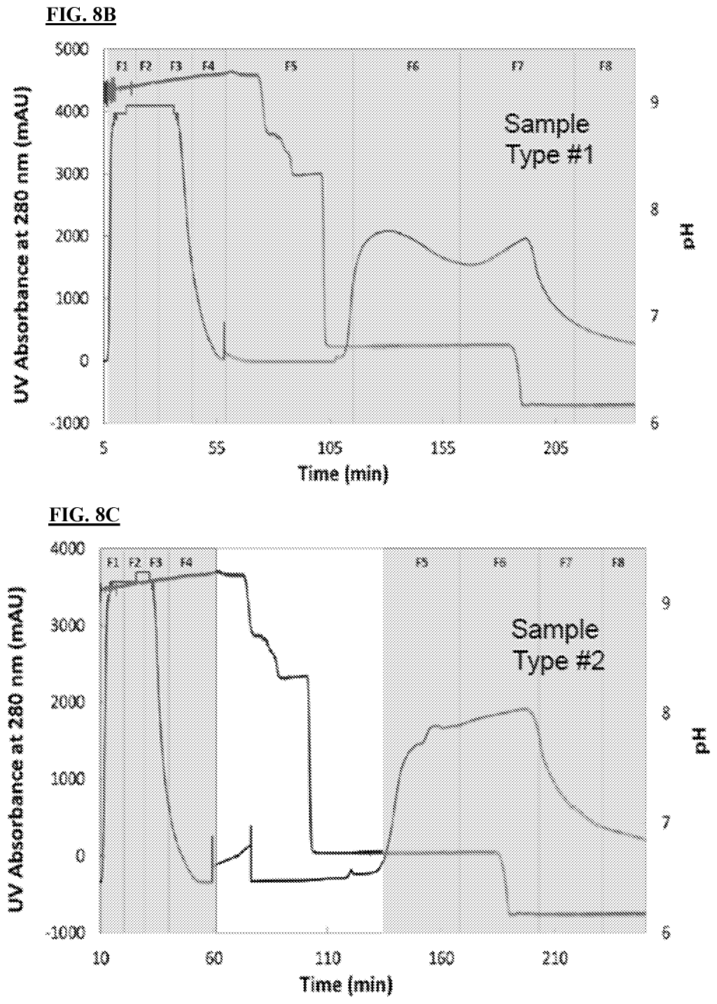

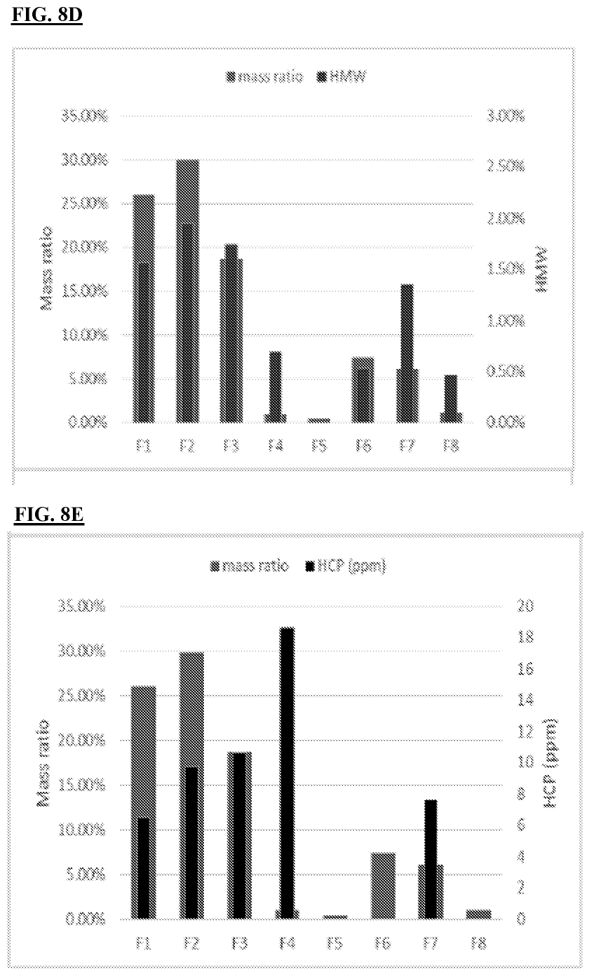

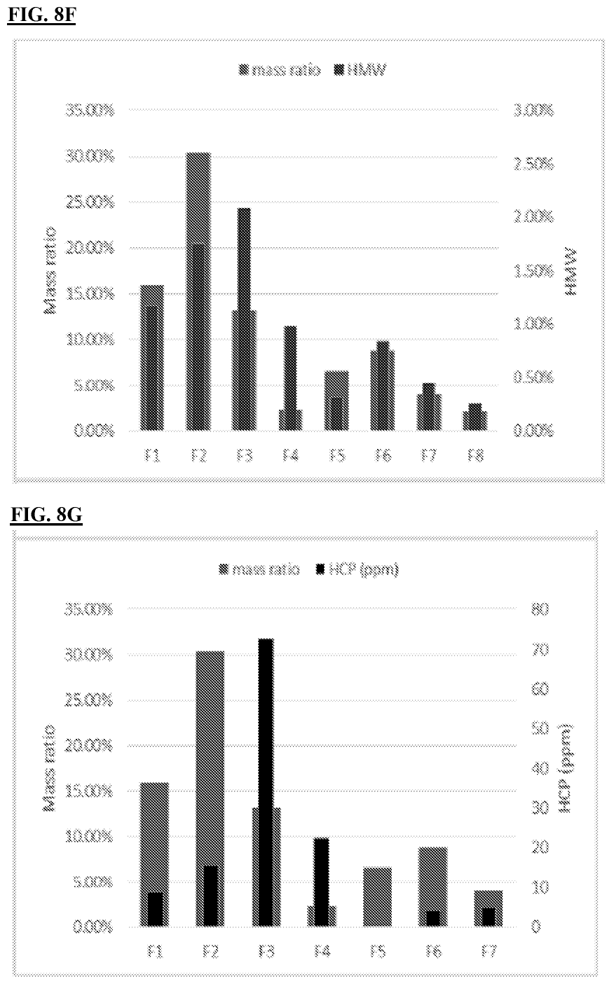

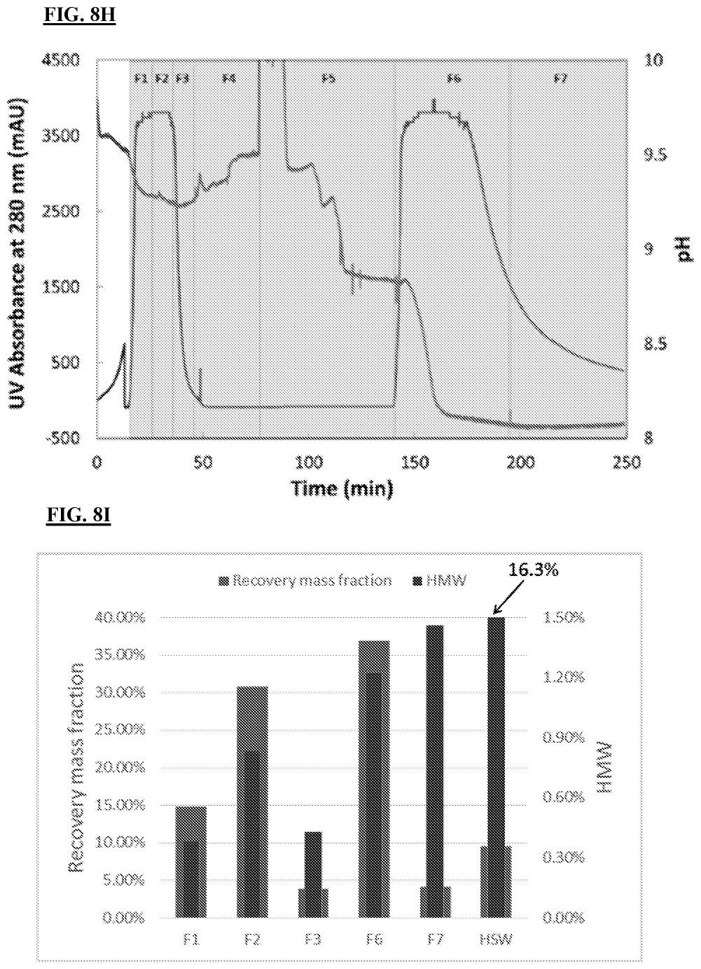

The feasibility of using the overload and elute mode with two equal size anion-exchange columns was tested, with column switching, in a chromatofocusing polishing step using a retained pH gradient, for antibodies from protein A pools (FIG. 8A). The results of anion-exchange chromatofocusing applied to the polishing of Antibody B from protein A pools using the overload and elute mode and two equal size Super Q columns, with column switching, is shown in FIGS. 8B-8G. Relative amounts HMWs and HCPs are shown in FIGS. 8D-8G. The properties of the columns and feed sample used are summarized in Table 6.

TABLE 6

| | | | | Feed | | |
|---|---|---|---|---|---|---|
| | | | | material | | |
| | | | Mass | HCP | Feed | |
| Sample | Number of | Column | loaded | amount | material | |
| type | columns | size | (mg) | (ppm) | HMW | FIG. |
| #1 | two | 0.7 mL + | 154 | 67 | 2.6% | FIGS. 8B, |
| | | 0.7 mL | | | | 8D, 8E |
| #2 | two | 0.7 mL + | 94 | 1316 | 4.2% | FIGS. 8C, |
| | | 0.7 mL | | | | 8F, 8G |

The results of anion-exchange chromatofocusing applied to the polishing of Antibody D from a protein A pool using the overload and elute mode and two equal size GigaCap columns, with column switching, is shown in FIGS. 8H-8I. Relative amounts HMWs are shown in FIG. 8I. The properties of the columns and feed sample used are summarized in Table 7.

TABLE 7

| | | | | Feed | | |
|---|---|---|---|---|---|---|
| | | | | material | | |
| | | | Mass | HCP | Feed | |
| Sample | Number of | Column | loaded | amount | material | |
| type | columns | size | (mg) | (ppm) | HMW | FIG. |
| #1 | two | 0.7 mL + | 129 | 6 | 2.4% | FIGs. 8H, 8I |
| | | 0.7 mL | | | | |

### Example 7

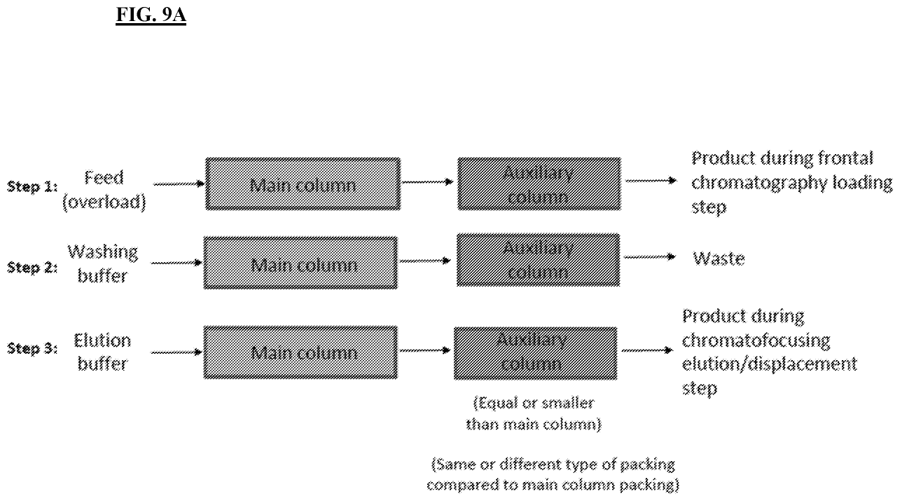

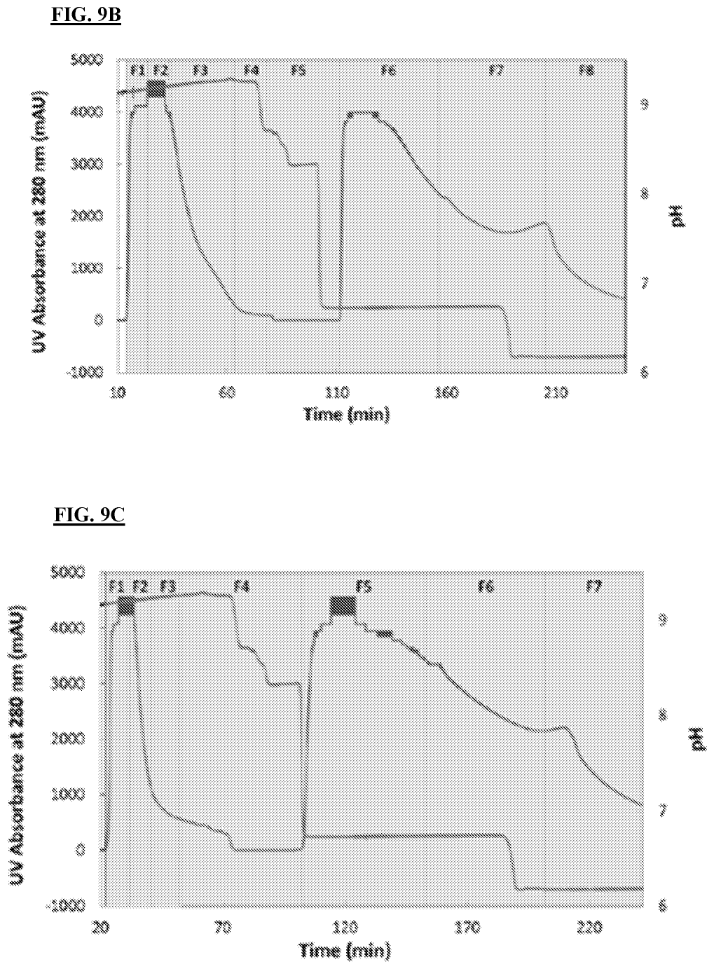

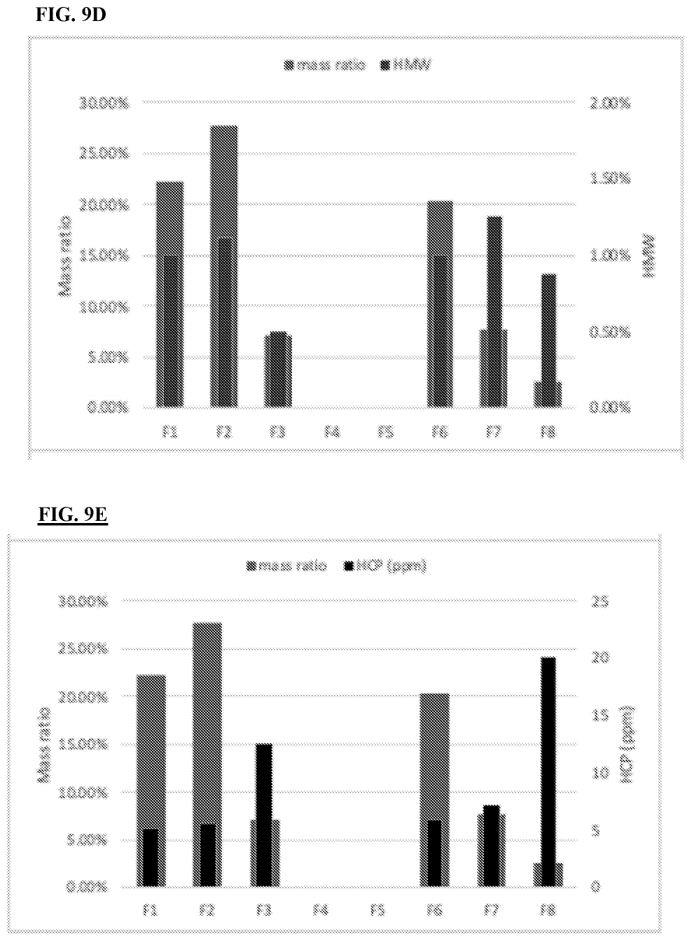

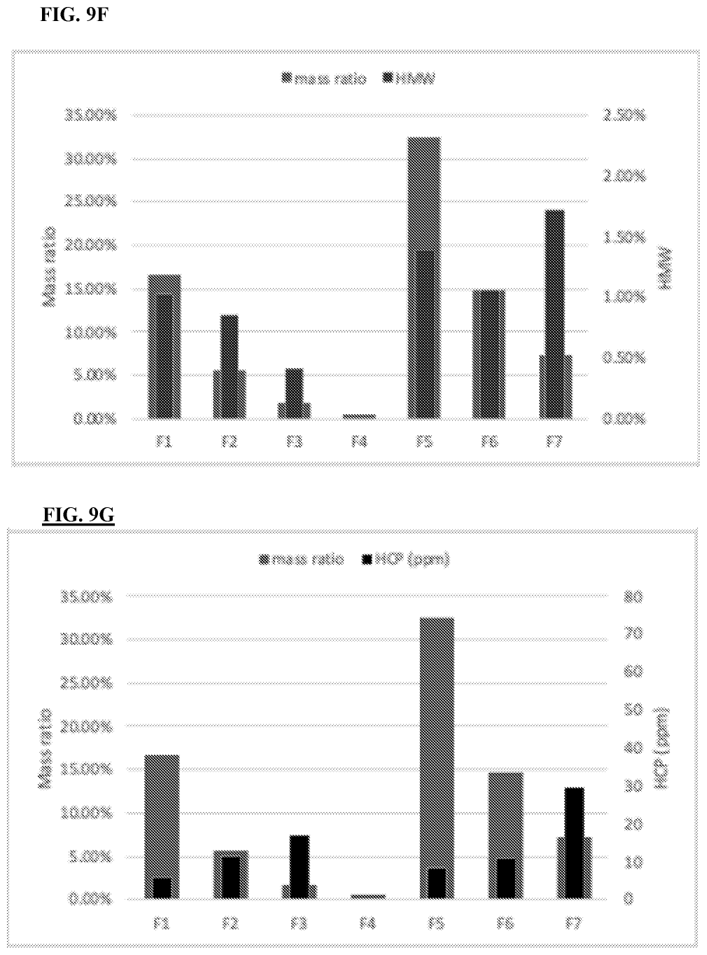

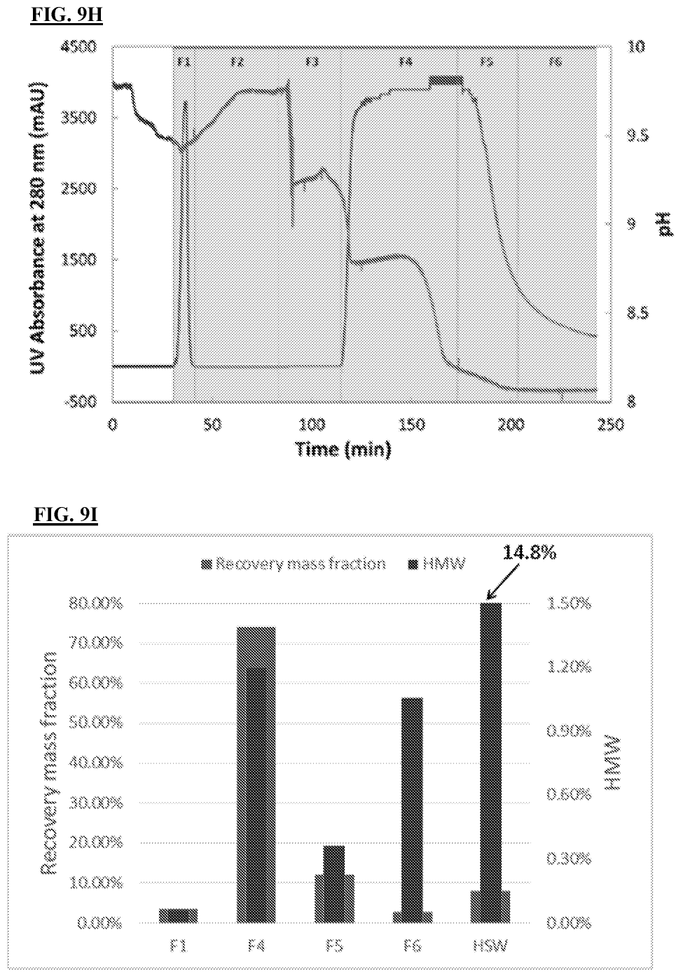

The feasibility of using the overload and elute mode with two equal size anion-exchange columns was tested, without column switching, in a chromatofocusing polishing step using a retained pH gradient, for antibodies from protein A pools (FIG. 9A). The results of anion-exchange chromatofocusing applied to the polishing of Antibody B from a protein A pool using the overload and elute mode with two equal size Super Q columns, without column switching, are shown in FIGS. 9B-9G. Relative amounts HMWs and HCPs are shown in FIGS. 9D-9G. The properties of the columns and feed sample used are summarized in Table 8.

TABLE 8

| | | | | Feed | | |
|---|---|---|---|---|---|---|
| | | | | material | | |
| | | | Mass | HCP | Feed | |
| Sample | Number of | Column | loaded | amount | material | |
| type | columns | size | (mg) | (ppm) | HMW | FIG. |
| #1 | two | 0.7 mL + | 125 | 46 | 2.3% | FIGS. 9B, |
| | | 0.7 mL | | | | 9D, 9E |
| #2 | two | 0.7 mL + | 104.6 | 1508 | 3.9% | FIGS. 9C, |
| | | 0.7 mL | | | | 9F, 9G |

The results of anion-exchange chromatofocusing applied to the polishing of Antibody D from a protein A pool using the overload and elute mode using two equal size GigaCap columns, without column switching, is shown in FIGS. 9H-9I. Relative amounts HMWs are shown in FIG. 9I. The properties of the columns and feed sample used are summarized in Table 9.

TABLE 9

| | | | | Feed | | |
|---|---|---|---|---|---|---|
| | | | | material | | |
| | | | Mass | HCP | Feed | |
| Sample | Number of | Column | loaded | amount | material | Corresponding |
| type | columns | size | (mg) | (ppm) | HMW | FIGS. |
| #1 | two | 0.7 mL + | 114 | 6 | 2.4% | 9H, 9I |
| | | 0.7 mL | | | | |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications, patents, and patent applications mentioned in this specification are herein incorporated by reference to the same extent as if each individual publication, patent, or patent application was specifically and individually indicated to be incorporated by reference.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of separating a protein product from a contaminant in a mixture, comprising contacting the mixture with a polishing chromatography matrix.

2. A method of polishing a protein product in a mixture comprising the protein product and a contaminant comprising contacting the mixture with a polishing chromatography matrix.

3. The method of claim 1 or 2, wherein the polishing chromatography matrix is used together with a retained gradient.

4. A method of separating a protein product from a contaminant in a mixture, comprising:
(1) contacting the mixture with a polishing chromatography matrix, and
(2) applying a retained gradient to the polishing chromatography matrix.

5. A method of polishing a protein product from a contaminant in a mixture, comprising
(1) contacting the mixture with a polishing chromatography matrix, and
(2) applying a retained gradient to the polishing chromatography matrix.

6. The method of any one of claims 1 to 5, wherein the mixture is applied to the polishing chromatography matrix at a loading amount that is equal to the product dynamic binding capacity (DBC) of the polishing chromatography matrix.

7. The method of any one of claims 1 to 5, wherein the mixture is applied to the polishing chromatography matrix at a loading amount that is less than the product dynamic binding capacity (DBC) of the polishing chromatography matrix.

8. The method of any one of claims 1 to 5, wherein the mixture is applied to the polishing chromatography matrix at a loading amount that exceeds the dynamic binding capacity (DBC) of the polishing chromatography matrix.

9. The method of any one of claims 1 to 8, wherein the protein product comprises a monoclonal antibody or an antigen binding fragment thereof (“mAb”) or an Fc fusion protein.

10. The method of any one of claims 1 to 9, wherein the contaminant associates with the polishing chromatography matrix.

11. The method of any one of claims 3 to 10, wherein the retained gradient comprises a pH gradient.

12. The method of any one of claims 3 to 11, wherein the retained gradient comprises a retained ionic strength gradient.

13. The method of any one of claims 1 to 5 and 8 to 12, wherein the loading amount of the mixture that is applied to the polishing chromatography matrix is at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold higher than the DBC of the polishing chromatography matrix.

14. The method of any one of claims 1 to 13, wherein the mixture comprises a product of a prior purification step.

15. The method of claim 14, wherein the prior purification step comprises applying a feed material to the prior purification step, wherein the feed material comprises a cell culture harvest, wherein the cell culture harvest comprises the protein product.

16. The method of claim 14 or 15, wherein the prior purification step comprises a protein bind and elute chromatography.

17. The method of any one of claims 14 to 16, wherein the prior purification step comprises an anion-exchange chromatography, a cation-exchange chromatography, an affinity chromatography, a mixed mode chromatography, a hydrophobic-interaction chromatography or any combination thereof.

18. The method of any one of claims 14 to 17, wherein the prior purification step comprises a protein A chromatography.

19. The method of any one of claims 1 to 18, wherein the polishing chromatography matrix comprises an anion-exchange matrix, a cation-exchange matrix, a mixed-mode matrix, a hydrophobic-interaction chromatography matrix or any combination thereof.

20. The method of any one of claims 1 to 19, wherein the polishing chromatography matrix comprises an anion-exchange matrix.

21. The method of any one of claims 1 to 20, wherein a starting buffer is applied to the polishing chromatography matrix.

22. The method of claim 21, wherein the starting buffer is applied to the polishing chromatography matrix prior to the contacting of the mixture comprising the protein product and the contaminant with the polishing chromatography matrix.

23. The method of claim 21 or 22, wherein the starting buffer comprises N-(1,1-Dimethyl-2-hydroxyethyl)-3-amino-2-hydroxypropanesulfonic acid (AMPSO), 3-morpholin-4-ylpropane-1-sulfonic acid (MOPS), or both.

24. The method of any one of claims 21 to 23, wherein the starting buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM AMPSO.

25. The method of any one of claims 21 to 24, wherein the starting buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, or about 10 mM to about 15 mM AMPSO.

26. The method of any one of claims 21 to 25, wherein the starting buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO.

27. The method of any one of claims 21 to 26, wherein the starting buffer comprises about 9.4 mM AMPSO.

28. The method of any one of claims 21 to 27, wherein the starting buffer comprises at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, or at least about 30 mM MOPS.

29. The method of any one of claims 21 to 28, wherein the starting buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS.

30. The method of any one of claims 21 to 29, wherein the starting buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS.

31. The method of any one of claims 21 to 30, wherein the starting buffer comprises about 20.5 mM MOPS.

32. The method of any one of claims 21 to 31, wherein the starting buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10.

33. The method of any one of claims 21 to 31, wherein the starting buffer has a pH of about 9.2.

34. The method of any one of claims 21 to 31, wherein the starting buffer comprises more than one buffering species, wherein each of the more than one buffering species travels through the matrix at a different velocity, thereby establishing a retained gradient.

35. The method of any one of claims 3 to 34, wherein the pH of the retained gradient is determined based on the apparent isoelectric points of the protein product and the contaminant.

36. The method of claim 35, wherein the apparent isoelectric points of the protein product and the contaminant are determined using a 2D analytical fractionation process.

37. The method of claim 36, wherein the 2D analytical fractionation process comprises subjecting the mixture comprising the protein product and the contaminant to an analytical chromatography using a retained gradient with multiple pH fronts followed by high-sensitivity silver stained SDS-PAGE.

38. The method of any one of claims 1 to 37, further comprising washing the polishing chromatography matrix with a wash buffer after the mixture comprising the protein product and the contaminant is contacted with the polishing chromatography matrix.

39. The method of claim 38, wherein the wash buffer comprises AMPSO, MOPS, or both.

40. The method of claim 38 or 39, wherein the wash buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM AMPSO.

41. The method of claim 39 or 40, wherein the wash buffer comprises about 0.1 mM to about 20 mM, about 0.5 mM to about 15 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, or about 10 mM to about 15 mM AMPSO.

42. The method of any one of claims 38 to 41, wherein the wash buffer comprises about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM AMPSO.

43. The method of any one of claims 38 to 42, wherein the wash buffer comprises about 9.4 mM AMPSO.

44. The method of any one of claims 38 to 43, wherein the wash buffer comprises at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, at least about 26 mM, at least about 27 mM, at least about 28 mM, at least about 29 mM, or at least about 30 mM MOPS.

45. The method of any one of claims 38 to 43, wherein the wash buffer comprises about 0.1 mM to about 30 mM, about 1 mM to about 25 mM, about 5 mM to about 25 mM, about 10 mM to about 30 mM, about 10 mM to about 25 mM, about 15 mM to about 30 mM, about 15 mM to about 25 mM, about 16 mM to about 24 mM, about 17 mM to about 23 mM, about 18 mM to about 22 mM, or about 19 mM to about 21 mM MOPS.

46. The method of any one of claims 38 to 45, wherein the wash buffer comprises about 19.0 mM, about 19.1 mM, about 19.2 mM, about 19.3 mM, about 19.4 mM, about 19.5 mM, about 19.6 mM, about 19.7 mM about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM MOPS.

47. The method of any one of claims 38 to 46, wherein the wash buffer comprises about 20.5 mM MOPS.

48. The method of any one of claims 38 to 47, wherein the wash buffer has a pH of at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, or at least about 10.

49. The method of any one of claims 38 to 48, wherein the wash buffer has a pH of about 9.2.

50. The method of any one of claims 38 to 49, wherein the wash buffer comprises more than one buffering species, wherein each of the more than one buffering species travels through the matrix at a different velocity, thereby establishing a retained gradient.

51. The method of any one of claims 38 to 50, wherein the wash buffer is the same as the starting buffer.

52. The method of any one of claims 3 to 51, wherein the retained gradient is created by performing a stepwise change between the starting buffer and an elution buffer.

53. The method of claim 52, wherein the elution buffer comprises MOPS, 4-Morpholineethanesulfonic acid monohydrate (MES), or both.

54. The method of any one of claims 52 to 53, wherein the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, or at least about 20 mM MOPS.

55. The method of any one of claims 53 to 56, wherein the elution buffer comprises about 0.1 mM to about 20 mM, about 0.5 mM to about 15 mM, about 5 mM to about 15 mM, about 6 mM to about 15 mM, about 7 mM to about 15 mM, about 8 mM to about 15 mM, about 9 mM to about 15 mM, about 10 mM to about 15 mM, about 10 mM to about 14 mM, or about 11 mM to about 13 mM MOPS.

56. The method of any one of claims 52 to 55, wherein the elution buffer comprises about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, or about 13.0 mM MOPS.

57. The method of any one of claims 52 to 56, wherein the elution buffer comprises about 12 mM MOPS.

58. The method of any one of claims 52 to 57, wherein the elution buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM MES.

59. The method of any one of claims 52 to 57, wherein the elution buffer comprises about 0.1 mM to about 20 mM, about 0.5 mM to about 15 mM, about 5 mM to about 15 mM, about 6 mM to about 14 mM, about 7 mM to about 13 mM, about 8 mM to about 12 mM, about 9 mM to about 11 mM, about 5 mM to about 10 mM, or about 10 mM to about 15 mM MES.

60. The method of any one of claims 52 to 59, wherein the elution buffer comprises about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10.0 mM, about 10.1 mM, about 10.2 mM, about 10.3 mM, about 10.4 mM, about 10.5 mM, about 10.6 mM, about 10.7 mM, about 10.8 mM, about 10.9 mM, or about 11.0 mM MES.

61. The method of any one of claims 52 to 60, wherein the elution buffer comprises about 10 mM MES.

62. The method of any one of claims 52 to 61, wherein the elution buffer has a pH of less than about 8.0, less than about 7.9, less than about 7.8, less than about 7.7, less than about 7.6, less than about 7.5, less than about 7.4, less than about 7.3, less than about 7.2, less than about 7.1, less than about 7.0, less than about 6.9, less than about 6.8, less than about 6.7, less than about 6.6, less than about 6.5, less than about 6.4, less than about 6.3, less than about 6.2, less than about 6.1, less than about 6.0, less than about 5.9, less than about 5.8, less than about 5.7, less than about 5.6, less than about 5.5, less than about 5.4, less than about 5.3, less than about 5.2, or less than about 5.1.

63. The method of any one of claims 52 to 62, wherein the elution buffer has a pH of about 6.2.

64. The method of any one of claims 52 to 62, wherein the elution buffer comprises more than one buffering species, wherein the more than one buffering species flow through the matrix at different velocities, thereby establishing a retained gradient.

65. The method of claim 64, wherein at least one of the more than one buffering species of the elution buffer is different from the more than one buffering species of the starting buffer.

66. The method of any one of claims 21 to 65, wherein the starting buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min.

67. The method of any one of claims 21 to 66, wherein the starting buffer is applied to the polishing chromatography matrix at a volumetric flow rate corresponding to a superficial velocity of about 5.0 cm/min.

68. The method of any one of claims 21 to 67, wherein the starting buffer is applied to the polishing chromatography matrix for about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, or about 50 minutes.

69. The method of any one of claims 21 to 68, wherein the starting buffer is applied to the polishing chromatography matrix for about 40 minutes.

70. The method of any one of claims 38 to 69, wherein the wash buffer is applied to the polishing chromatography matrix at a volumetric flow rate corresponding to a superficial liquid velocity of at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min, at least about 4.0 cm/min, at least about 4.1 cm/min, at least about 4.2 cm/min, at least about 4.3 cm/min, at least about 4.4 cm/min, at least about 4.5 cm/min, at least about 4.6 cm/min, at least about 4.7 cm/min, at least about 4.8 cm/min, at least about 4.9 cm/min, or at least about 5.0 cm/min.

71. The method of any one of claims 38 to 70, wherein the wash buffer is applied to the polishing chromatography matrix at a volume flow rate corresponding to a superficial velocity of about 5.0 cm/min.

72. The method of any one of claims 38 to 71, wherein the wash buffer is applied to the polishing chromatography matrix for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, 21 minute, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes.

73. The method of any one of claims 38 to 72, wherein the wash buffer is applied to the polishing chromatography matrix for about 10 minutes.

74. The method of any one of claims 38 to 72, wherein the wash buffer is applied to the polishing chromatography matrix for about 30 minutes.

75. The method of any one of claims 52 to 74, wherein the stepwise change between the starting buffer and the elution buffer is applied to the polishing chromatography matrix at a volumetric flow rate corresponding to a superficial liquid velocity of at least about 1.4 cm/min, at least about 1.5 cm/min, at least about 1.6 cm/min, at least about 1.7 cm/min, at least about 1.8 cm/min, at least about 1.9 cm/min, at least about 2.0 cm/min, at least about 2.1 cm/min, at least about 2.2 cm/min, at least about 2.3 cm/min, at least about 2.4 cm/min, at least about 2.5 cm/min, at least about 2.6 cm/min, at least about 2.7 cm/min, at least about 2.8 cm/min, at least about 2.9 cm/min, at least about 3.0 cm/min, at least about 3.1 cm/min, at least about 3.2 cm/min, at least about 3.3 cm/min, at least about 3.4 cm/min, at least about 3.5 cm/min, at least about 3.6 cm/min, at least about 3.7 cm/min, at least about 3.8 cm/min, at least about 3.9 cm/min or at least about 4.0 cm/min.

76. The method of any one of claims 38 to 75, wherein the stepwise change between the starting buffer and the elution buffer is applied to the polishing chromatography matrix at a volume flow rate corresponding to a superficial velocity of about 2.8 cm/min.

77. The method of any one of claims 1 to 76, comprising a second polishing chromatography matrix, wherein material eluted from the polishing chromatography matrix following the contacting of the mixture with the polishing chromatography column matrix is applied to a second polishing chromatography matrix.

78. The method of claims 1 to 77, wherein a column switching method is employed for overload and elution after the mixture is contacted with the polishing chromatography matrix, wherein an auxiliary column containing a second polishing chromatography matrix is inserted into the mobile phase flow path downstream from the polishing chromatography matrix during elution.

79. The method of any one of claims 21 to 78, wherein the starting buffer comprises at least about 16 mM, at least about 17 mM, at least about 18 mM, at least about 19 mM, or at least about 20 mM AMPSO.

80. The method of claim 79, wherein the starting buffer comprises about 19.0 mM, about 19.1 mM, about 19.2 mM, about 19.3 mM, about 19.4 mM, about 19.5 mM, about 19.6 mM, about 19.7 mM, about 19.8 mM, about 19.9 mM, about 20.0 mM, about 20.1 mM, about 20.2 mM, about 20.3 mM, about 20.4 mM, about 20.5 mM, about 20.6 mM, about 20.7 mM, about 20.8 mM, about 20.9 mM, or about 21.0 mM AMPSO.

81. The method of claim 79 or 80, wherein the starting buffer comprises about 20.5 mM AMPSO.

82. The method of any one of claims 79 to 81, wherein the starting buffer comprises at least about 6 mM, at least about 7 mM, at least about 8 mM, at least about 9 mM, or at least about 10 mM glycine.

83. The method of any one of claims 79 to 82, wherein the starting buffer comprises about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, or about 10.0 mM glycine.

84. The method of any one of claims 79 to 83, wherein the starting buffer comprises about 9.4 mM glycine.

85. The method of any one of claims 79 to 84, wherein the starting buffer has a pH of at least about 9.0, at least about 9.1, at least about 9.2, at least about 9.3, at least about 9.4, at least about 9.5, at least about 9.6, at least about 9.7, at least about 9.8, at least about 9.9, or at least about 10.

86. The method of any one of claims 79 to 86, wherein the starting buffer has a pH of about 9.8.

87. The method of any one of claims 79 to 86, wherein the starting buffer comprises about 9.4 mM glycine and about 20.5 mM AMPSO, at about pH 9.8.

88. The method of any one of claims 52 to 87, wherein the elution buffer comprises about 11.0 mM, about 11.5 mM, about 12.0 mM, about 12.5 mM, or about 13.0 mM AMPSO.

89. The method of claim 88, wherein the elution buffer comprises about 12 mM AMPSO.

90. The method of any one of claims 52 to 89, wherein the elution buffer comprises about 9.0 mM, about 9.5 mM, about 10.0 mM, about 10.5 mM, or about 11.0 mM tricine.

91. The method of claim 90, wherein the elution buffer comprises about 10 mM tricine.

92. The method of any one of claims 52 to 91, wherein the elution buffer has a pH of at least about 7.5, at least about 7.6, at least about 7.7, at least about 7.8, at least about 7.9, at least about 8.0, or at least about 8.1.

93. The method of any one of claims 89 to 92, wherein the elution buffer has a pH of about 8.07.

94. The method of any one of claims 52 to 90, wherein the elution buffer comprises about 12 mM AMPSO and about 10 mM tricine, at about pH 8.07.
